(19) United States
(12) Patent Application Publication
(10) Pub. No.: US 2023/0129100 A1
(43) Pub. Date: Apr. 27, 2023

(54) DETECTION OF LOW ABUNDANCE NUCLEIC ACIDS

(71) Applicant: BioSkryb Genomics, Inc., Durham, NC (US)

(72) Inventors: Charles GAWAD, Durham, NC (US); Jay A.A. WEST, Durham, NC (US); Siddhartha KADIA, Durham, NC (US)

(21) Appl. No.: 17/907,575

(22) PCT Filed: May 5, 2021
(86) PCT No.: PCT/US2021/030837

§ 371 Date: Sep. 28, 2022

Related U.S. Application Data
(60) Provisional application No. 63/021,477, filed on May 7, 2020.

(51) Int. Cl. C12Q 1/6853; C12Q 1/6806; C12N 15/10

(52) U.S. Cl. CPC C12Q 1/6853; C12Y 207/07007; C12Q 1/6806; C12N 15/1093

(57) ABSTRACT

Provided herein are compositions and methods for detecting nucleic acids. Further provided are methods using Primary Template-Directed Amplification (PTA) to detect trace nucleic acids. Such methods in some instances are applied to diagnostics, biotechnology and pharmaceutical manufacturing, and food safety.

### CROSS-REFERENCE TO RELATED APPLICATIONS

This application claims the benefit of U.S. Provisional Application No. 63/021,477 filed May 7, 2020, which is incorporated herein by reference for all purposes in its entirety.

### BACKGROUND

Nucleic acid detection is an important process in medicine, industry, and research fields. For example, trace nucleic acid detection provides information regarding the spread of viruses, or provides important controls for biotechnology which may suffer from nucleic acid contamination. Biologic drugs (e.g., drugs synthesized from recombinant expression in host cells) require high purity which are free form extraneous nucleic acids. There exists a need for improved methods to detect these trace nucleic acids.

### BRIEF SUMMARY

Provided herein are methods of detecting the presence or absence of trace nucleic acids comprising: providing a sample from a source, wherein the source comprises no more than 1 nanogram of nucleic acids; contacting the sample with at least one amplification primer, at least one strand displacement polymerase, and a mixture of nucleotides to generate replication products; and measuring a signal obtained from the replication products, wherein the signal to a noise ratio (SNR) greater than 1.01 indicates the sample comprises trace nucleic acids. Further provided herein are methods wherein the sample comprises no more than 0.1 nanograms of nucleic acids. Further provided herein are methods wherein the sample comprises no more than 1 picograms of nucleic acids. Further provided herein are methods wherein the sample comprises no more than 1 femtograms of nucleic acids. Further provided herein are methods wherein the nucleic acids comprise no more than 50 million nucleosides. Further provided herein are methods wherein the nucleic acids comprise no more than 100,000 nucleosides. Further provided herein are methods wherein the nucleic acids comprise no more than 10,000 nucleosides. Further provided herein are methods wherein the nucleic acids have an average length of 200-2000 bases. Further provided herein are methods wherein the nucleic acids have an average length of at least 1000 bases. Further provided herein are methods wherein the method further comprises establishing a noise amount from a no template control experiment. Further provided herein are methods wherein contacting occurs for no more than 10 hours. Further provided herein are methods wherein contacting occurs for no more than 4 hours. Further provided herein are methods wherein contacting occurs for no more than 2 hours. Further provided herein are methods wherein the ratio of the signal to the noise is greater than 1000. Further provided herein are methods wherein a SNR greater than 1.05 indicates the sample comprises trace nucleic acids. Further provided herein are methods wherein a SNR greater than 1.1 indicates the sample comprises trace nucleic acids. Further provided herein are methods wherein the signal is fluorescence, phosphorescence, chemiluminescence, or colorimetric. Further provided herein are methods wherein the nucleic acids comprise nucleic acids derived from bacteria, yeast, fungi, molds, insect, or human sources. Further provided herein are methods wherein the sample is obtained from one or more of an enzyme-containing reagent, a pharmaceutical composition, a boot or carcass swab, blood, hair, skin, saliva, and a human clinical isolate. Further provided herein are methods wherein the sample further comprises proteins. Further provided herein are methods wherein the proteins are recombinantly expressed proteins. Further provided herein are methods wherein the sample comprises at least one nucleic acid, and the at least one nucleic acid is amplified in step b). Further provided herein are methods wherein the amplification is performed under substantially isothermic conditions. Further provided herein are methods wherein the amplification is performed under conditions wherein the temperature varies by no more than 10 degrees C. Further provided herein are methods wherein the amplification is performed under conditions wherein the temperature varies by no more than 5 degrees C. Further provided herein are methods wherein the nucleic acid polymerase is a DNA polymerase. Further provided herein are methods wherein the DNA polymerase is a strand displacing DNA polymerase. Further provided herein are methods wherein the nucleic acid polymerase is bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase. Further provided herein are methods wherein the nucleic acid polymerase does not comprise 3′->5′ exonuclease activity. Further provided herein are methods wherein the polymerase is Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, or Therminator DNA polymerase. Further provided herein are methods wherein the mixture of nucleotides comprises at least one terminator nucleotide which terminates nucleic acid replication by the strand displacement polymerase. Further provided herein are methods wherein the nucleic acid polymerase comprises 3′->5′ exonuclease activity and the at least one terminator nucleotide inhibits the 3′->5′ exonuclease activity. Further provided herein are methods wherein the least one terminator nucleotide comprises modifications of the r group of the 3′ carbon of the deoxyribose. Further provided herein are methods wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof. Further provided herein are methods wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are methods wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are methods wherein the nucleotides with modification to the alpha group are alpha-thio dideoxynucleotides. Further provided herein are methods wherein the amplification primers are 4 to 70 nucleotides in length. Further provided herein are methods wherein the at least one amplification primer is 4 to 20 nucleotides in length. Further provided herein are methods wherein the at least one amplification primer comprises a randomized region. Further provided herein are methods wherein the randomized region is 4 to 20 nucleotides in length. Further provided herein are methods wherein the randomized region is 8 to 15 nucleotides in length. Further provided herein are methods wherein the amplification products are between about 50 and about 2000 nucleotides in length. Further provided herein are methods wherein the amplification products are between about 200 and about 1000 nucleotides in length. Further provided herein are methods wherein the amplification proceeds for 5-15 cycles. Further provided herein are methods wherein the amplification proceeds for no more than 20 cycles. Further provided herein are methods wherein the method further comprises qPCR. Further provided herein are methods wherein at least one amplification primer comprises a cleavable fluorophore and quencher. Further provided herein are methods wherein the method comprises at least four amplification primers. Further provided herein are methods wherein the method further comprises contacting the sample with a single-stranded DNA binding protein. Further provided herein are methods wherein the method further comprises contacting the sample with a helicase. Further provided herein are methods wherein the method further comprises contacting the sample with a nicking enzyme. Further provided herein are methods wherein the method further comprises contacting the sample with a reverse transcriptase. Further provided herein are methods wherein the method further comprises quantifying the concentration of nucleic acids in the sample. Further provided herein are methods wherein the method further comprises discarding or repurifying a sample which is found to contain trace nucleic acids.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a comparison of the PTA (Primary Template-directed Amplification)-Irreversible Terminator method with a different embodiment, namely the Reversible Terminator method. Terminators control the length and number of primary template amplicons. Reversible terminators may be removed by polymerases with exonuclease activity, which allows further amplification of primary template amplicons.

FIG. 2 illustrates a graph of signal vs. amplification cycles using the PTA method to amplify varying amounts of starting NA12878 DNA template. The y-axis contains delta-Rn values of 1×10³ to 1×10⁷ at factor 10 intervals. The x-axis contains cycle numbers from 0 to 120 at 5 cycle intervals, and corresponding times of 0 to 600 at 25 minute intervals.

FIG. 3A illustrates amplified nucleic acid yields using PTA from buccal swab samples for bacterial genomic DNA. NTC: no template control. The 100 pg and 50 pg samples represent mixed gram negative and gram positive cultures. SC1-SC8 are gram positive cells, SC9-SC16 are gram negative cells. The y-axis depicts the yield in nanograms from 0 to 4500 at 500 ng intervals.

FIG. 3B illustrates a gel showing the size of amplicons obtained from the PTA reaction.

FIG. 3C illustrates a graph of the lengths of the longest contigs in representative samples obtained from buccal samples.

FIG. 3D illustrates a graph of the lengths of the longest contigs in the bulk gDNA obtained from buccal samples.

FIG. 3E illustrates a graph mapping contigs to bacterial genre. Samples mapped predominantly to two genera of Enterobacteriaceae (both were Gram-negative).

FIG. 4A illustrates a plate x-y alignment for FACS sorting of DH5a E. coli samples.

FIG. 4B illustrates cultures of DH5α E. coli prior to FACS sorting. Left tube: no inoculation, LB media only. Middle tube: 3 microliters DH5α E. coli inoculation (1:1000), OD₆₀₀=0.047; Right tube: 30 microliters DH5α bacteria inoculation (1:100), OD₆₀₀=0.078.

FIGS. 4C-4E illustrate FACS sorting results of DH5α E. coli after staining with Syto9 (stains both gram positive and gram negative), hexidium iodide (stains gram positive), and propidium iodide (“PI”, stains dead cells). FIG. 4C illustrates FACS sorting of DH5α E. coli (no stain control). Left: all events. middle: cells. right: PI negative. FIG. 4D illustrates FACS sorting of DH5α E. coli (dead cell control). Left: all events. middle: cells. right: cells. FIG. 4E illustrates FACS sorting of DH5α bacteria (index sort). Left: all events. middle: cells. right: cells. Sort included both PI and live cells.

FIG. 4F illustrates a map of the plate obtained from FACS sorting of DH5α E. coli. Wells contained nothing (control, column 1), 2 microliters bacterial cell buffer only (column 2), 3 microliters bacterial cell buffer+a single cell (columns 3-11), 3 microliters bacterial cell buffer+5 cells (column 12, rows A-D), and microliters bacterial cell buffer only (column 12, rows E-H).

FIG. 5A illustrates yields (ng) of amplified DNA after PTA (replicate 1) for no template control (NTC) samples, single cells SC1-SC24, and five cell wells (5C1/5C2) obtained from FACS sorting of DH5α E. coli.

FIG. 5B illustrates a gel of amplicon sizes obtained from the PTA reaction (replicate 1) for DH5α E. coli cells.

FIG. 6A illustrates yields (ng) of amplified DNA after PTA (replicate 2) for no template control (NTC) samples, single cells SC1-SC24, and five cell wells (5C1/5C2) obtained from FACS sorting of DH5α E. coli.

FIG. 6B illustrates a gel of amplicon sizes obtained from the PTA reaction (replicate 2) for DH5α E. coli cells.

FIGS. 7A-7B illustrate gels showing DNA fragment sizes obtained from DH5α E. co/i or buccal swab samples. Yields were approximately 7 ng/microliter with fragments of approximately 450 bases in length. FIG. 7A illustrates a gel of DNA libraries obtained from DH5α E. coli cells. FIG. 7B illustrates a gel of DNA libraries obtained from buccal swab samples.

FIG. 8A illustrates a graph of DH5α E. coli contig assembly of the library shown in FIG. 7A. Most samples have contigs of approximately 100 kb in length. gDNA had the largest contigs. The y-axis contains lengths (bases) of 0 to 260,000, in 20,000 base intervals. Sequence numbers are shown along the x-axis.

FIG. 8B illustrates a graph of contig mapping to species. Most samples had nearly identical taxon distribution, and samples all map highest to E. coli at family level. The 100 pg control comprised a small proportion of Staphylococcaceae.

FIGS. 9A-9B illustrate FACS sorting results of B. subtilis after staining with Syto9 (stains both gram positive and gram negative), hexidium iodide (stains gram positive), and propidium iodide (“PI”, stains dead cells). FIG. 9A illustrates FACS sorting of B. subtilis (no stain control). Upper left: all events. Upper right: cells. Lower left: PI negative. Lower right: dead. FIG. 9B illustrates FACS sorting of B. subtilis (index sort). Upper left: all events. Upper right: cells. Lower left: PI negative.

FIG. 9C illustrates a gel of amplicon sizes obtained from the PTA reaction for B. subtilis cells.

FIG. 9D illustrates a gel of amplicon sizes obtained from a prepared library of B. subtilis cells.

FIG. 9E illustrates yields (ng) of amplified DNA after PTA (replicate 1) on no template control (NTC) samples, single cells SC1-SC24, and five cell wells (5C1/5C2) obtained from FACS sorting of B. subtilis.

FIG. 10 illustrate FACS sorting results of a mixed DH5α E. coli/B. subtilis population after staining with Syto9 (stains both gram positive and gram negative), hexidium iodide (stains gram positive), and propidium iodide (“PI”, stains dead cells). Upper left: all events. Upper right: cells. Lower left: PI negative. Lower right: dead.

### DETAILED DESCRIPTION OF THE INVENTION

There is a need to develop new scalable, accurate and efficient methods for nucleic acid detection which would overcome limitations in the current methods by increasing accuracy, and sensitivity. For example, present methods such as MDA (multiple strand displacement) in some instances result in high background levels for small quantities of DNA. These high backgrounds may be caused by reagent impurities, contamination during setup, or non-specific amplification. Such reactions may in some cases utilize “no template” controls (NTCs) to measure and identify these sources of background observed. Described herein are methods comprising “Primary Template-Directed Amplification” (PTA) which facilitate rapid detection of small quantities of nucleic acids. Further provided herein are methods of nucleic acid detection utilizing PTA to establish one or more NTCs are free from nucleic acid contamination. Further provided herein are methods utilizing PTA for the detection of nucleic acids on surfaces.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/of” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A practical Approach, Volumes I and II (D. N. Glover ed. 1985); Oligonucleotide Synthesis (MJ. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, viral DNA, viral RNA, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, and Kivioja, T. et al. Nat. Methods (2012) 9: 72-74.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes.

The term “identity” or “homology” refer to the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Conservative substitutions in some instances involve substitution of one amino acid of similar shape (e.g., tyrosine for phenylalanine) or charge (glutamic acid for aspartic acid) for another. A polynucleotide or polynucleotide region (or a peptide or peptide region) comprises a certain percentage (for example, 80%, 85%, 90%, or 95%) of “sequence identity” or “homology” to another sequence means that, when aligned, that percentage of bases (or amino acids) are the same in comparing the two sequences. Neither N- or C-terminal extensions nor insertions shall be construed as reducing identity or homology. Alignment and the percent homology or sequence identity in some instances are determined using software programs known by those skilled the art. In some instances, default parameters are used for alignment. An exemplary alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Similarity, or percent similarity in some instances of two sequences is the sum of both identical and similar matches (residues that have undergone conservative substitution). In some instances, similarity is measured using the program BLAST “Positives.”

The term signal to noise ratio (“SNR”) is some instances refers to a ratio between a measured or generated signal and a noise value. In some instances, a noise value is the amount of signal measured in the absence of an analyte (e.g., nucleic acid). In some instances, a noise value is the amount of signal measured in a no template control (NTC) experiment. In some instances, a noise value is the amount of signal measured in an experiment which does not comprise a polymerase.

Methods of Nucleic Acid Detection

Described herein are methods of detecting trace or low abundance nucleic acids. In some instances, nucleic acids are detected using PTA. In some instances, detection of the presence or absence of nucleic acids comprises obtaining a sample from a source, amplifying nucleic acids (if present) using at least one strand displacing polymerase, at least one primer, a mixture of nucleotides, and obtaining a signal correlated to the number of nucleic acids in the sample. In some instances, the mixture of nucleotides comprises at least one terminator which prevents amplification of at least one amplicon. In some instances, at least some primers are random. In some instances, primers are configured to bind to specific sequences, such as specific nucleic acids to be detected. Signals in some instance are obtained from fluorescence, phosphorescence, chemiluminescence, or colorimetric signals. Multiple channels of signal are in some instances measured simultaneously. In some instances, at least 1, 2, 3, 4, 5, or more than 6 channels are measured. In some instances, each channel comprises a unique or partially unique signal on the electromagnetic spectrum. Optionally, methods described herein are used in conjunction with additional amplification methods such as PCR, RPA, LAMP, HDA (helicase-dependent amplification), NEAR (nicking enzyme amplification reaction), or other amplification method. In some instances, a signal to noise ratio (SNR) is used to determine the presence or absence of nucleic acids in the sample. If a signal obtained from the methods described herein meets or exceeds a pre-determined threshold, the sample is in some instances determined to contain nucleic acids. Conversely, an SNR below this level in some instances is determined to be free of nucleic acids. Additionally, methods described herein also allow for accurate determination of the concentration of nucleic acids in the sample.

Signals may be acquired through any method know in the art. In some instances, a fluorescent signal is obtained after amplification by PTA. In some instances a fluorescence signal is measured directly by use of a spectrophotometer (e.g., Nanodrop, Qubit instrument). Signals may be acquired by colorimetric analysis. Such methods comprise use of dyes, such as pH sensitive dyes, which change color in response to the nucleic acid concentration in the sample. These dyes in some instances are used to measure the rate of an amplification reaction, or measure when a reaction has reached a pre-determined conversion. In some instances, a chemical or biological reaction generates nucleic acids products, and is monitored by methods described herein. In some instances, reaction monitoring comprises use of intercalating dyes. In some instances, reaction monitoring comprises use of dye-linked polynucleotides. Non-limiting examples of dyes include phenol red, cresol red, neutral red, and m-cresol purple, or other dye.

The signal to noise ratio (SNR) for methods described herein may be used to establish the presence or absence of nucleic acids in a sample. In some instances, an SNR of at least 1.01, 1.05, 1.10, 1.2, 1.5, 2, 3, 5, 10, 20, or at least 50 indicates the presence of nucleic acids in the sample. In some instances, an SNR of 1.01-1.5, 1.01-1.2, 1.01-1.1, 1.05-1.10, 1.05-1.5, 1-3, 1-5, 1-10, or 1.1-10 50 indicates the presence of nucleic acids in the sample. In some instances, a noise value is determined by comparison to an analogous analysis of a sample which does not comprise any nucleic acids. In some instances, a noise value is determined by comparison to an analogous analysis of a sample which does not comprise specific nucleic acids which are being detected (e.g., no template control).

Methods described herein may detect trace or low abundance nucleic acids. In some instances, the amount of nucleic acids which may be detected is measured by mass. In some instances, samples comprise no more than 1, 2, 5, 10, 20, 50, 80, 100, 200, 500, 800, 1000, or 1200 nanograms of nucleic acids. In some instances, samples comprise no more than 1, 2, 5, 10, 20, 50, 80, 100, 200, 500, 800, 1000, or 1200 femtograms of nucleic acids. In some instances, samples comprise no more than 0.01, 0.02, 0.05, 0.1, 0.2, 0.5, 0.8, 1, or 1.2 femtograms of nucleic acids. In some instances, the amount of nucleic acids which may be detected is measured by number of molecules. In some instances, samples comprise no more than 1, 2, 5, 8, 10, 20, 50, 80, 100, 120, 150, 180, 200, or no more than 220 molecules of nucleic acids. In some instances, samples comprise no more than 100, 200, 500, 1000, 2000, 5000, 10000, 20000, 50000, 100,000, 200,000, 500,000, or no more than 1 million molecules of nucleic acids. In some instances, the amount of nucleic acids which may be detected is measured by number of nucleotides. In some instances, samples comprise no more than 100, 200, 500, 1000, 2000, 5000, 10000, 20000, 50000, 100,000, 200,000, 500,000, or no more than 1 million nucleotides.

Detection may be defined as a measured signal greater than a background (noise) or control signal. In some instances, detection is defined as a normalized reporter value (ΔRn). In some instances, a copy number (cp) represents the number of nucleic acid molecules present in a sample. In some instances, the reporter value is obtained from a fluorescent signal. In some instances, the normalized reporter value is calculated as the experimental signal value minus the background signal. In some instances, the normalized reporter value is calculated as the experimental signal value minus the control signal. In some instances, a method described herein produces a normalized reporter value of at least 0.01, 0.02, 0.03, 0.04, 0.05, 0.06 or at least 0.07. In some instances, a method described herein produces a normalized reporter value of at least 0.01, 0.02, 0.03, 0.04, 0.05, 0.06 or at least 0.07, for a sample comprising at least 1 cp. In some instances, a method described herein produces a normalized reporter value of at least 0.01, 0.02, 0.03, 0.04, 0.05, 0.06 or at least 0.07, for a sample comprising at least 2 cp. In some instances, a method described herein produces a normalized reporter value of at least 0.01, 0.02, 0.03, 0.04, 0.05, 0.06 or at least 0.07, for a sample comprising at least 5 cp. In some instances, a method described herein produces a normalized reporter value of at least 0.01, 0.02, 0.03, 0.04, 0.05, 0.06 or at least 0.07, for a sample comprising at least 8 cp. In some instances, a method described herein produces a normalized reporter value of at least 0.01, 0.02, 0.03, 0.04, 0.05, 0.06 or at least 0.07, for a sample comprising at least 10 cp. In some instances, a method described herein produces a normalized reporter value of at least 0.01, 0.02, 0.03, 0.04, 0.05, 0.06 or at least 0.07, for a sample comprising at least 20 cp. In some instances, a method described herein produces a normalized reporter value of at least 0.01, 0.02, 0.03, 0.04, 0.05, 0.06 or at least 0.07, for a sample comprising at least 1 cp and subjected to no more than 60 cycles. In some instances, a method described herein produces a normalized reporter value of at least 0.01, 0.02, 0.03, 0.04, 0.05, 0.06 or at least 0.07, for a sample comprising at least 1 cp and subjected to no more than 50 cycles. In some instances, a method described herein produces a normalized reporter value of at least 0.01, 0.02, 0.03, 0.04, 0.05, 0.06 or at least 0.07, for a sample comprising at least 1 cp and subjected to no more than 45 cycles. In some instances, a method described herein produces a normalized reporter value of at least 0.01, 0.02, 0.03, 0.04, 0.05, 0.06 or at least 0.07, for a sample comprising at least 2 cp and subjected to no more than 40 cycles. In some instances, a method described herein produces a normalized reporter value of at least 0.01, 0.02, 0.03, 0.04, 0.05, 0.06 or at least 0.07, for a sample comprising at least 5 cp and subjected to no more than 38 cycles. In some instances, a method described herein produces a normalized reporter value of at least 0.01, 0.02, 0.03, 0.04, 0.05, 0.06 or at least 0.07, for a sample comprising at least 8 cp and subjected to no more than 36 cycles. In some instances, a method described herein produces a normalized reporter value of at least 0.01, 0.02, 0.03, 0.04, 0.05, 0.06 or at least 0.07, for a sample comprising at least 10 cp and subjected to no more than 34 cycles. In some instances, a method described herein produces a normalized reporter value of at least 0.01, 0.02, 0.03, 0.04, 0.05, 0.06 or at least 0.07, for a sample comprising at least 20 cp and subjected to no more than 32 cycles. In some instances, cycles are obtained through isothermal amplification (e.g., PTA method). In some instances, the method comprises amplification of a genomic or fragment thereof in the presence of at least one terminator nucleotide, wherein the number of amplification cycles is less than 70, 60, 50, 40, 30, 20, 15, 12, 10, 9, 8, 7, 6, 5, 4, or less than 3 cycles. In some instances, the average length of amplification products is 100-1000, 200-500, 200-700, 300-700, 400-1000, or 500-1200 bases in length. In some instances, the method comprises amplification of a genomic or fragment thereof in the presence of at least one terminator nucleotide, wherein the number of amplification cycles is no more than 6 cycles. In some instances, the at least one terminator nucleotide does comprise a detectable label or tag. In some instances, the amplification comprises 2, 3, or 4 terminator nucleotides. In some instances, at least two of the terminator nucleotides comprise a different base. In some instances, at least three of the terminator nucleotides comprise a different base. In some instances, four terminator nucleotides each comprise a different base. The number of direct copies may be controlled in some instances by the number of amplification cycles. In some instances, no more than 70, 60, 50, 40, 30, 25, 20, 15, 13, 11, 10, 9, 8, 7, 6, 5, 4, or 3 cycles are used to generate copies of the target nucleic acid molecule. In some instances, about 70, 60, 50, 40, 30, 25, 20, 15, 13, 11, 10, 9, 8, 7, 6, 5, 4, or about 3 cycles are used to generate copies of the target nucleic acid molecule. In some instances, about 3, 4, 5, 6, 7, 8, 10, 15, 20, 25, 30, 35, 40, 50, 60, or about 70 cycles are used to generate copies of the target nucleic acid molecule. In some instances, 2-4, 2-5, 2-7, 2-8, 2-10, 2-15, 3-5, 3-10, 3-15, 4-10, 4-15, 5-10, 5-15, 5-70, 10-70, 20-70, 30-70, 40-70, 50-70, 10-20, 10-30, or 10-40 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

In some instances, such methods comprise one or more steps in a workflow. For example, in a first step a sample (e.g., biological sample) is acquired from a source. In some instances, a source is a patient, surface, or other source. In a second step, the sample is extracted to isolate nucleic acids. In a third step, the extracted nucleic acids are assayed or identified to establish if they comprise nucleic acids of a virus. In a fourth step, results of the assay are reported to a healthcare provider, patient, electronic display, or electronic database.

Described herein are methods for nucleic acid detection from sources. Such methods in some instances comprise at least the steps of sample acquisition, and sample assay. In some instances, methods described herein comprise at least the steps of sample acquisition, sample assay, and reporting. In some instances, methods described herein are capable of multiplexing, wherein multiple samples are analyzed in parallel.

Samples may be acquired from any source which may contain nucleic acids. Such samples in some instances are utilized during a sample acquisition step. In some instances, a source includes but is not limited to a fluid (e.g., water source, bodily fluid), gas (air sample), or solid (medical surface, mask). In some instances, a source is a fluid. In some instances, the fluid is obtained from an animal. In some instances, the animal is a mammal. In some instances, the mammal is a human. Samples in some instances are obtained from blood, serum, plasma, bone marrow, urine, saliva, mucus, cerebrospinal fluid, pleural fluid, pericardial fluid, ascites, or aqueous humor. In some instances, samples are obtained from upper or lower respiratory sources. In some instances, sources include but are not limited to nasopharyngeal or oropharyngeal swabs, sputum, lower respiratory tract aspirates, bronchoalveolar lavage, nasopharyngeal wash/aspirate, or nasal aspirate. In some instances, sample sources comprise surfaces. In some instances, surfaces include but are not limited to animal carcasses, floors, walls, medical devices, or other surface which is suspected to comprise nucleic acids. In some instances, samples comprise indwelling medical devices, such as but not limited to, intravenous catheters, urethral catheters, cerebrospinal shunts, prosthetic valves, artificial joints, or endotracheal tubes. In some instances samples are obtained from swabs of a surface. In some instances, a surface comprises the respiratory tract, nose, ear, throat, lung, or esophagus.

Samples may be obtained from sources where nucleic acids are scarce or presumed to absent of nucleic acids. In some instances, a source comprises samples obtained from a “clean room”. Such sources include but are not limited to clean rooms for manufacturing (e.g., biotechnology reagents, semiconductors, pharmaceuticals), space exploration equipment, extraplanetary samples, and medical/surgery suites. In some instances, clean room particle counts are acquired by particle counting instruments, such as an aerosol particle counter. In some instances, a particle counter is an optical particle counter. In some instances, a particle counter is a condensation particle counter. In some instances a clean room comprise a standardized ISO 14644-type clean room. In some instances a clean room comprise a standardized ISO 14644-1 class 1, 2, 3, 4, 5, 6, 7, 8, or class 9 clean room. In some instances a clean room comprise a standardized ISO 5295 class 1, 2, 3, or class 4 clean room. In some instances, a clean room comprises no more than 1,000,000, 100,000, 10,000, 1,000, 100, or no more than 10 particles per cubic meter, wherein the particles are at least 0.1 micron in size. In some instances, a clean room comprises no more than 237,000, 23,700, 2,370, 237, 24, or no more than 2 particles per cubic meter, wherein the particles are at least 0.2 micron in size. In some instances, a clean room comprises no more than 102,000, 10,200, 1,020, 102, or no more than 10 particles per cubic meter, wherein the particles are at least 0.3 micron in size. In some instances, a clean room comprises no more than 35,200,000, 3,520,000, 352,000, 35,200, 3,520, 352, 35, or no more than 4 particles per cubic meter, wherein the particles are at least 0.5 micron in size. In some instances, a clean room comprises no more than 8,320,000, 832,000, 83,200, 8,320, 832, 83, or no more than 8 particles per cubic meter, wherein the particles are at least 1 micron in size. In some instances, a clean room comprises no more than 294,000, 29,300, 2,930, 293, or no more than 29 particles per cubic meter, wherein the particles are at least 5 micron in size.

Extraction steps may be used to purify nucleic acids prior to a sample assay step. In some instances, methods described herein to not comprise an extraction step. In some instances, methods described herein comprise no more than 4, 3, 2, or 1 extraction steps. In some instances, a method described herein does not comprise an extraction step. In some instances, a method described herein does not comprise binding nucleic acids to a solid support, precipitating nucleic acids, or ion-exchange chromatography. In some instance, extraction steps include cell lysis, nucleic acid binding, washing bound nucleic acids, drying bound nucleic acids, and eluting bound nucleic acids. In some instances, extraction steps comprise binding a nucleic acid to a solid support. In some instances, extract steps comprise precipitating a nucleic acid. In some instances, extraction steps comprise hybridizing a nucleic acid to an array. In some instances In some instances, extraction comprises binding nucleic acids to a solid support. In some instances, extraction comprises use of beads (e.g., SPRI beads). In some instances, extraction comprises use of ion-exchange chromatography. In some instances, a workflow is limited to extracting 5-10, 5-100, 12-96, 24-64, 8-96, 48-96, or 48-72 samples in a single batch. In some instances, one or more extraction steps is completed in 10-240 min, 10-180 min, 10-120 min, 90-180 min, 120-180 min, 60-180 min, or 120-300 min. In some instances, one or more extraction steps is completed in at least 10 min, 20 min, 30 min, 45 min, 60 min, 90 min, 120 min, 180 min, or at least 240 min.

One or more additional steps may precede a sample assay step. In some instances, methods described herein comprise treatment of a sample with a lysis buffer prior to an assay step. In some instances, a lysis buffer comprises a proteinase. In some instances, the proteinase is proteinase K or Pk. In some instances, the lysis buffer is stored as a lyophilized powder. In some instances, the lyophilized powder comprises a stabilizer. In some instances, the stabilizer is a sugar. In some instances, the sugar is selected from maltose, trehalose, cellobiose, sucralose, isomaltose, raffinose, or isomaltulose. In some instances, a stabilizer is present at about 1%, 2%, 5%, 10%, 15%, 20%, 30%, 50%, or about 75% (w/w). In some instances, a stabilizer is present at 1-5%, 1-20%, 5-20%, 10-50%, 20-50%, or 15-30% (w/w). In some instances, a lysis buffer comprises a reducing agent. In some instances, the reducing agent is DTT or beta-mercaptoethanol. In some instances, the lysis buffer comprises a surfactant. In some instances, a sample is heat treated prior to an assay step. In some instances, a sample is heated at one or more temperatures, each for a period of time. In some instances, heating the sample deactivates one or more enzymes in the sample, such as RNAses. In some instances, a sample is heated to a first temperature for a first time, and then heated at a second temperature for a second time. In some instances, the first temperature is 25-75 deg C., 25-60 deg C., 25-50 deg C., 25-40 deg C., 30-45 deg C., 35-45 deg C., 35-50 deg C., or 30-60 deg C. In some instances, the first temperature is about 25 deg C., 30 deg C., 32 deg C., 35 deg C., 37 deg C., 39 deg C., 40 deg C., 42 deg C., 45 deg C., 50 deg C., 55 deg C., or about 60 deg C. In some instances, the second temperature is 65-95 deg C., 65-90 deg C., 65-85 deg C., 65-80 deg C., 70-95 deg C., 75-90 deg C., 78-84 deg C., or 80-90 deg C. In some instances, the first temperature is about 60 deg C., 65 deg C., 70 deg C., 75 deg C., 80 deg C., 85 deg C., 90 deg C., 95 deg C., about 98 deg C. In some instances, the first time is 5-30 min, 10-20 min, 5-20 min, 8-13 min, or 15-30 min. In some instances, the first time is about 5 min, 8 min, 10 min, 12 min, 15 min, 17 min, 20 min, 30 min, or 45 min. In some instances, the second time is 5-30 min, 10-20 min, 5-20 min, 8-13 min, or 15-30 min. In some instances, the second time is about 5 min, 8 min, 10 min, 12 min, 15 min, 17 min, 20 min, 30 min, or 45 min. In some instances, the first temperature is 25-75 deg C., 25-60 deg C., 25-50 deg C., 25-40 deg C., 30-45 deg C., 35-45 deg C., 35-50 deg C., or 30-60 deg C. and the first time is 10-20 min. In some instances, the first temperature is about 25 deg C., 30 deg C., 32 deg C., 35 deg C., 37 deg C., 39 deg C., 40 deg C., 42 deg C., 45 deg C., 50 deg C., 55 deg C., or about 60 deg C. and the first time is about 15 min. In some instances, the second temperature is 65-95 deg C., 65-90 deg C., 65-85 deg C., 65-80 deg C., 70-95 deg C., 75-90 deg C., 78-84 deg C., or 80-90 deg C. and the first time is 10-20 min. In some instances, the first temperature is about 60 deg C., 65 deg C., 70 deg C., 75 deg C., 80 deg C., 85 deg C., 90 deg C., 95 deg C., about 98 deg C. and the first time is about 15 min.

Sample assays may be used to detect the presence of bacterial, fungal, or viral particles. Various viral particles may be detected using a sample assay. Such detections in some instances comprise use of target-specific primers which are configured to bind to viral nucleic acid sequences. In some instances, viral particles comprise nucleic acids. In some instances, nucleic acids comprise DNA or RNA. In some instances, nucleic acids comprise RNA. In some instances, an assay step comprises analysis of a positive control. In some instances, a positive control comprises nucleic acids associated with a virus. In some instances, a positive control comprises RNA. In some instances, a positive control comprises DNA. In some instances, a positive control comprises a plasmid. In some instances, a positive control is generated in-situ. In some instances, an assay step comprises a negative control (no template control). In some instances, a negative control does not comprise viral nucleic acids. In some instances, an assay step comprises analysis of a positive control and a negative control. Positive controls in some instances are specific to a specific type of virus. In some instances, a positive control is a COVID-19 plasmid. In some instances, a positive control comprises an RNA copy of a viral gene. In some instances, viral genes include but are not limited to N1, N2, and/or N3. In some instances, a control targeting human RNaseP is used to establish a sample comprises at least some nucleic acids for testing, regardless of whether it comprises viral nucleic acids. In some instances, a negative sample control (without sample) is used to establish if any cross-contamination has occurred between samples. In some instances, a virus is detected by the presence of one or more different nucleic acids. In some instances, a sample assay is completed in about 10 min, 20 min, 30 min, 45 min, 60 min, 90 min, 120 min, or about 180 min. In some instances, a sample assay is completed in no more than 10 min, 20 min, 30 min, 45 min, 60 min, 90 min, 120 min, or no more than 180 min. In some instances, a sample assay is completed in 10 min-180 min 10-120 min, 10-60 min, 10-30 min, 30-180 min, 30-120 min, 60-120 min, 60-90 min, 90-120 min, or 45-100 min.

Sample assays may comprise one or more reporter assays to quantify target organisms via detection of nucleic acids. In some instances, sample assays comprise a probe comprising a recognition moiety and a reporter moiety. In some instances, a recognition moiety binds to an organism component, such as a viral nucleic acid, bacterial nucleic acid, or other organism (or fragment thereof). In some instances, a reporter moiety generates a signal which indicates the presence of a viral nucleic acid. In some instances, a reporter moiety generates a signal which indicates the presence of a bacterial nucleic acid. In some instances, a reporter moiety generates a signal which indicates the presence of a food-borne pathogen. In some instances, signals include but are not limited to fluorescence, phosphorescence, chemiluminescence, antibody/antigen binding, radioactivity, mass tags, next generation sequencing, or other detectable signal. In some instances, sample assays comprise use of a polymerase chain reaction. In some instances, sample assays comprise a reverse transcriptase. In some instances, sample assays comprise a polymerase. In some instances, sample assays comprise quantitative polymerase chain reactions (qPCR), or real-time PCR. In some instances, sample assays comprise quantitative reverse-transcriptase polymerase chain reactions (qRT-PCR). In some instances, a sample assay step comprises use of one or more primers, such as a forward primer and a reverse primer. In some instances, the amount of nucleic acid in a sample is quantified after one or more PCR cycles. In some instances, a sample assay step comprises about 1, 2, 5, 10, 12, 15, 18, 20, 25, 30, 35, 40, or about 45 PCR cycles. In some instances, a sample assay step comprises no more than 1, 2, 5, 10, 12, 15, 18, 20, 25, 30, 35, 40, or no more than 45 PCR cycles. In some instances, sample assays comprise reverse transcription of RNA into cDNA. In some instances, sample assay steps comprise binding of a reporter moiety to a target nucleic acid (e.g., viral or bacterial nucleic acids). In some instance, a probe comprises a quenching moiety. In some instances, a probe comprises a nucleic acid complementary to a nucleic acid. Any number of probes are in some instances used during a sample assay step. In some instances a sample assay comprises at least two probes. In some instances a first probe is configured to bind a first nucleic acid, and a second probe is configured to bind a control (non-target organism) nucleic acid. In some instances, a control nucleic acid is a human gene or fragment thereof.

Sample assays utilizing PTA may result in significant advantages over other amplification methods. In some instances, a longer amplification time (e.g., more cycles) required to detect smaller amounts of nucleic acids does not lead to a signal in the absence of such nucleic acids. In some instances, PTA results in higher quality amplicon products, which are more easily sequenced and identified. In some instances, PTA results in more accurate quantification of trace nucleic acids in a sample. In some instances, the presence or absence of a nucleic acid is determined in no more than 12, 10, 8, 6, 5, 4, 3, 2, or 1 hour. In some instances, a sample which does not comprise nucleic acids does not produce significant amounts of detectable product. In some instances, “no detectable product” comprises the amount of signal obtained from an assay, without the addition of any nucleic acids (primers or otherwise). In some instances, a sample which does not comprise nucleic acids does not produce significant amounts of detectable product after no more than 14, 12, 10, 8, 6, 4, 3, 2, or 1 hour.

Sample assays may comprise use of droplet-based PCR, wherein each PCR reaction occurs in a droplet. In some instances, sample assays comprise use digital PCR (dPCR). In some instances, dPCR is used to detect single molecules. In some instances, signals obtained from dPCR include reporter-labeled primers, or intercalating dyes.

Sample assays may comprise use of exponential amplification methods. In some instances, assays comprise use of MDA. In some instances, MDA-based assays comprise a plurality of primers, and an isothermal strand-displacing polymerase. In some instances, primers comprise randomized sequences. In some instances, one or more primers comprise a 3′ thiophosphate. In some instances, sample assays comprise use of three or more primers. In some instances, sample assays use nested primers. In some instances, MDA-based amplification assays proceed for no more than 4 hours, 3 hours, 2 hours, 1 hour, 30 min, or nor more than 15 minutes. In some instances, sample assays comprise use of quasi-exponential amplification methods. In some instances, MDA is used for assays wherein the amount of nucleic acids targeted for detection is at least 1, 2, 5, 10, 50, 100, 500, 1000, 5000, or more than 5000 picograms of nucleic acids.

Sample assays may comprise loop-mediated isothermal amplification (LAMP). Sample assays in some instances comprise reverse-transcriptase loop-mediated isothermal amplification (RT-LAMP). In some instances, a sample assay comprises use of an isothermal polymerase. In some instances, a sample assay comprises use of an isothermal polymerase and a reverse transcriptase. In some instances, each PCR cycle during LAMP is held at a relatively constant temperature, for example 45-50 deg C., 50-55 deg C., 55-60 deg C., 60-65 deg C., 65-70 deg C., or 70-75 deg C. In some instances, primers used in sample assays comprise loop primers (primers comprising intramolecular loops). In some instances the assay readout includes colorimetric detection. In some instances, nested primers are used with the methods described herein. In some instances, at least 4 primers are used for amplification.

Nested primers may be used to control amplification, and may be used with any of the amplification methods described herein (e.g., PTA, MDA, LAMP, etc.). Such methods in some instances reduce non-specific priming and increase the number of desired amplicon products. For example, a sample comprises one or more nucleic acids, wherein at least some of the nucleic acids comprise a target nucleic acid region. In some instances, three or more primers are configured to amplify the target nucleic acid region. In some instances, primers are defined by sets of two. In some instances, a first set of primers (forward and reverse) are configured to hybridize to a first region nearer the 5′ end of the target nucleic acid and a second region nearer the 3′ end of the target nucleic acid (“inner primers”). In some instances, a second set of primers are configured to hybridize to a third region nearer the 5′ end of the target nucleic acid and a second region nearer the 3′ end of the target nucleic acid, wherein the second set of primers is not configured to bind to an amplicon generated by the first set of primers (“outer primers”). Any number of sets of primers may be used in some instances, for example 1, 2, 3, 4, 5, 6, 7, or more than 7 sets of primers. In some instances, each progressive set of primers is configured to bind a greater distance from the target nucleic acid region than the previous set of primers. In some instances, a set of “inner” primers generate a smaller amplicon than a set of “outer” of primers. In some instances, different sets of primers are added at different times during an amplification. In some instances, primer sets configured to generate longer amplicons are added earlier than primer sets configured to generate shorter amplicons. In some instances, the concentration of primers in a set is varied to control the rate of amplification. In some instances, the concentration of the first set of primers (“inner primers”) is higher than the concentration of the second set of primers (or additional sets of primers). In some instances the ratio of the concentration between two sets of primers is about 2:1, 5:1, 10:1, 50:1, 100:1, 500:1, 1000:1, 5000:1, or 10,000:1. In some instances, one or more nested primers comprise RNA. In some instances, RNA-comprising primers are used in conjunction with RNase to facilitate primer displacement.

Sample assays may comprise reverse transcriptase PTA (RT-PTA). Sample assays in some instances comprise an RT-PCR reaction to generate cDNA, followed by use of the PTA method to amplify the cDNA library. Such libraries are then sequenced, for example, using Next Generation Sequencing to detect the presence of viral nucleic acids.

Sample assays may comprise reverse transcriptase RPA (RT-RPA). Sample assays in some instances comprise an RT-RPA reaction to generate cDNA, followed by use of the RPA method to amplify the cDNA library and detect the viral genome using a primer and probe. In some instances, RPA comprises use of a recombinase, a single stranded DNA binding protein (SSB), and a strand-displacing enzyme. In some instances, each PCR cycle in RPA is held at a relatively constant temperature, for example 30-50 deg C., 30-55 deg C., 35-45 deg C., 30-40 deg C., 35-40 deg C., or 37-42 deg C.

Methods described herein may be used to detect pathogens. In some instances, pathogens are of bacteria, fungal, or viral origin. In some instances, the pathogen is a food-borne pathogen. Food-borne pathogens include but are not limited to Campylobacter, Clostridium botulinum, E. coli (e.g., E. coli O157:H7), Listeria monocytogenes, Norovirus, Salmonella, Staphylococcus aureus, Shigella, Toxoplasma gondii, hepatitis A, or Vibrio vulnificus. In some instances, a pathogen is a parasite.

Methods described herein may be used to detect viruses, virus particles, or other viral component or sub-component of a virus. In some instances, viruses comprise respiratory viruses. Virus include but are not limited to influenza or a coronavirus. In some instances, virus possesses hemagglutinin activity. In some instances, the virus is capable of infecting mammalian cells. In some instances, the virus is capable of infecting erythrocytes. In some instances, the coronavirus comprises SARS, MERS, Covid-19, bovine, norovirus, orthoreoviruses (reoviruses), human rotaviruses, human coronaviruses, adenoviruses, filoviruses, or other coronavirus. In some instances, the coronavirus is SARS. In some instances, the coronavirus is Covid-19. In some instances, the coronavirus is a bovine coronavirus. In some instances, the coronavirus is norovirus. In some instances, the coronavirus is an orthoreoviruses (e.g., reoviruses). In some instances, the coronavirus is a human rotaviruses, In some instances, the coronavirus is a human coronaviruses. In some instances, the coronavirus is an adenovirus. In some instances, the influenza is selected from avian flu, swine flu, or other flu.

In some instances, the virus is Abelson leukemia virus; Abelson murine leukemia virus; Abelson's virus; Acute laryngotracheobronchitis virus; Adelaide River virus; Adeno associated virus group; Adenovirus; African horse sickness virus; African swine fever virus; AIDS virus; Aleutian mink disease parvovirus; Alpharetrovirus; Alphavirus; ALV related virus; Amapari virus; Aphthovirus; Aquareovirus; Arbovirus; Arbovirus C; arbovirus group A; arbovirus group B; Arenavirus group; Argentine hemorrhagic fever virus; Argentine hemorrhagic fever virus; Arterivirus; Astrovirus; Ateline herpesvirus group; Aujezky's disease virus; Aura virus; Ausduk disease virus; Australian bat lyssavirus; Aviadenovirus; avian erythroblastosis virus; avian infectious bronchitis virus; avian leukemia virus; avian leukosis virus; avian lymphomatosis virus; avian myeloblastosis virus; avian paramyxovirus; avian pneumoencephalitis virus; avian reticuloendotheliosis virus; avian sarcoma virus; avian type C retrovirus group; Avihepadnavirus; or Avipoxvirus; B virus.

In some instances, Babanki virus; baboon herpesvirus; baculovirus; Barmah Forest virus; Bebaru virus; Berrimah virus; Betaretrovirus; Birnavirus; Bittner virus; BK virus; Black Creek Canal virus; bluetongue virus; Bolivian hemorrhagic fever virus; Boma disease virus; border disease of sheep virus; borna virus; bovine alphaherpesvirus 1; bovine alphaherpesvirus 2; bovine coronavirus; bovine ephemeral fever virus; bovine immunodeficiency virus; bovine leukemia virus; bovine leukosis virus; bovine mammillitis virus; bovine papillomavirus; bovine papular stomatitis virus; bovine parvovirus; bovine syncytial virus; bovine type C oncovirus; bovine viral diarrhea virus; Buggy Creek virus; bullet shaped virus group; Bunyamwera virus supergroup; Bunyavirus; Burkitt's lymphoma virus; or Bwamba Fever.

In some instances, the virus is CA virus; Calicivirus; California encephalitis virus; camelpox virus; canarypox virus; canid herpesvirus; canine coronavirus; canine distemper virus; canine herpesvirus; canine minute virus; canine parvovirus; Cano Delgadito virus; caprine arthritis virus; caprine encephalitis virus; Caprine Herpes Virus; Capripox virus; Cardiovirus; caviid herpesvirus 1; Cercopithecid herpesvirus 1; cercopithecine herpesvirus 1; Cercopithecine herpesvirus 2; Chandipura virus; Changuinola virus; channel catfish virus; Charleville virus; chickenpox virus; Chikungunya virus; chimpanzee herpesvirus; chub reovirus; chum salmon virus; Cocal virus; Coho salmon reovirus; coital exanthema virus; Colorado tick fever virus; Coltivirus; Columbia SK virus; common cold virus; contagious eethyma virus; contagious pustular dermatitis virus; Coronavirus; Corriparta virus; coryza virus; covid-19 (Wuhan virus); cowpox virus; coxsackie virus; CPV (cytoplasmic polyhedrosis virus); cricket paralysis virus; Crimean-Congo hemorrhagic fever virus; croup associated virus; Cryptovirus; Cypovirus; Cytomeg Finalovirus; cytomegalovirus group; or cytoplasmic polyhedrosis virus.

In some instances, the virus is deer papillomavirus; deltaretrovirus; dengue virus; Densovirus; Dependovirus; Dhori virus; diploma virus; Drosophila C virus; duck hepatitis B virus; duck hepatitis virus 1; duck hepatitis virus 2; duovirus; Duvenhage virus; or Deformed wing virus DWV.

In some instances, the virus is eastern equine encephalitis virus; eastern equine encephalomyelitis virus; EB virus; Ebola virus; Ebola-like virus; echo virus; echovirus; echovirus 10; echovirus 28; echovirus 9; ectromelia virus; EEE virus; EIA virus; EIA virus; encephalitis virus; encephalomyocarditis group virus; encephalomyocarditis virus; Enterovirus; enzyme elevating virus; enzyme elevating virus (LDH); epidemic hemorrhagic fever virus; epizootic hemorrhagic disease virus; Epstein-Barr virus; equid alphaherpesvirus 1; equid alphaherpesvirus 4; equid herpesvirus 2; equine abortion virus; equine arteritis virus; equine encephalosis virus; equine infectious anemia virus; equine morbillivirus; equine rhinopneumonitis virus; equine rhinovirus; Eubenangu virus; European elk papillomavirus; European swine fever virus; Everglades virus; or Eyach virus.

In some instances, the virus is felid herpesvirus 1; feline calicivirus; feline fibrosarcoma virus; feline herpesvirus; feline immunodeficiency virus; feline infectious peritonitis virus; feline leukemia/sarcoma virus; feline leukemia virus; feline panleukopenia virus; feline parvovirus; feline sarcoma virus; feline syncytial virus; Filovirus; Flanders virus; Flavivirus; foot and mouth disease virus; Fort Morgan virus; Four Corners hantavirus; fowl adenovirus 1; fowlpox virus; Friend virus; Gammaretrovirus; GB hepatitis virus; GB virus; German measles virus; Getah virus; gibbon ape leukemia virus; glandular fever virus; goatpox virus; golden shinner virus; Gonometa virus; goose parvovirus; granulosis virus; Gross' virus; ground squirrel hepatitis B virus; group A arbovirus; Guanarito virus; guinea pig cytomegalovirus; or guinea pig type C virus.

Hantaan virus; Hantavirus; hard clam reovirus; hare fibroma virus; HCMV (human cytomegalovirus); hemadsorption virus 2; hemagglutinating virus of Japan; hemorrhagic fever virus; hendra virus; Henipaviruses; Hepadnavirus; hepatitis A virus; hepatitis B virus group; hepatitis C virus; hepatitis D virus; hepatitis delta virus; hepatitis E virus; hepatitis F virus; hepatitis G virus; hepatitis nonA nonB virus; hepatitis virus; hepatitis virus (nonhuman); hepatoencephalomyelitis reovirus 3; Hepatovirus; heron hepatitis B virus; herpes B virus; herpes simplex virus; herpes simplex virus 1; herpes simplex virus 2; herpesvirus; herpesvirus 7; Herpesvirus ateles; Herpesvirus hominis; Herpesvirus infection; Herpesvirus saimiri; Herpesvirus suis; Herpesvirus varicellae; Highlands J virus; Hirame rhabdovirus; hog cholera virus; human adenovirus 2; human alphaherpesvirus 1; human alphaherpesvirus 2; human alphaherpesvirus 3; human B lymphotropic virus; human betaherpesvirus 5; human coronavirus; human cytomegalovirus group; human foamy virus; human gammaherpesvirus 4; human gammaherpesvirus 6; human hepatitis A virus; human herpesvirus 1 group; human herpesvirus 2 group; human herpesvirus 3 group; human herpesvirus 4 group; human herpesvirus 6; human herpesvirus 8; human immunodeficiency virus; human immunodeficiency virus 1; human immunodeficiency virus 2; human papillomavirus; human T cell leukemia virus; human T cell leukemia virus I; human T cell leukemia virus II; human T cell leukemia virus III; human T cell lymphoma virus I; human T cell lymphoma virus II; human T cell lymphotropic virus type 1; human T cell lymphotropic virus type 2; human T lymphotropic virus I; human T lymphotropic virus II; human T lymphotropic virus III; Ichnovirus; infantile gastroenteritis virus; infectious bovine rhinotracheitis virus; infectious haematopoietic necrosis virus; infectious pancreatic necrosis virus; influenza virus A; influenza virus B; influenza virus C; influenza virus D; influenza virus pr8; insect iridescent virus; insect virus; iridovirus; Japanese B virus; Japanese encephalitis virus; JC virus; Junin virus;

In some instances, the virus is Kaposi's sarcoma-associated herpesvirus; Kemerovo virus; Kilham's rat virus; Klamath virus; Kolongo virus; Korean hemorrhagic fever virus; kumba virus; Kysanur forest disease virus; Kyzylagach virus; La Crosse virus; lactic dehydrogenase elevating virus; lactic dehydrogenase virus; Lagos bat virus; Langur virus; lapine parvovirus; Lassa fever virus; Lassa virus; latent rat virus; LCM virus; Leaky virus; Lentivirus; Leporipoxvirus; leukemia virus; leukovirus; lumpy skin disease virus; lymphadenopathy associated virus; Lymphocryptovirus; lymphocytic choriomeningitis virus; or lymphoproliferative virus group.

In some instances, the virus is Machupo virus; mad itch virus; mammalian type B oncovirus group; mammalian type B retroviruses; mammalian type C retrovirus group; mammalian type D retroviruses; mammary tumor virus; Mapuera virus; Marburg virus; Marburg-like virus; Mason Pfizer monkey virus; Mastadenovirus; Mayaro virus; ME virus; measles virus; Menangle virus; Mengo virus; Mengovirus; Middelburg virus; milkers nodule virus; mink enteritis virus; minute virus of mice; MLV related virus; MM virus; Mokola virus; Molluscipoxvirus; Molluscum contagiosum virus; monkey B virus; monkeypox virus; Mononegavirales; Morbillivirus; Mount Elgon bat virus; mouse cytomegalovirus; mouse encephalomyelitis virus; mouse hepatitis virus; mouse K virus; mouse leukemia virus; mouse mammary tumor virus; mouse minute virus; mouse pneumonia virus; mouse poliomyelitis virus; mouse polyomavirus; mouse sarcoma virus; mousepox virus; Mozambique virus; Mucambo virus; mucosal disease virus; mumps virus; murid betaherpesvirus 1; murid cytomegalovirus 2; murine cytomegalovirus group; murine encephalomyelitis virus; murine hepatitis virus; murine leukemia virus; murine nodule inducing virus; murine polyomavirus; murine sarcoma virus; Muromegalovirus; Murray Valley encephalitis virus; myxoma virus; Myxovirus; Myxovirus multiforme; or Myxovirus parotitidis.

In some instances, the virus is Nairobi sheep disease virus; Nairovirus; Nanirnavirus; Nariva virus; Ndumo virus; Neethling virus; Nelson Bay virus; neurotropic virus; New World Arenavirus; newborn pneumonitis virus; Newcastle disease virus; Nipah virus; noncytopathogenic virus; Norwalk virus; nuclear polyhedrosis virus (NPV); nipple neck virus; O'nyong'nyong virus; Ockelbo virus; oncogenic virus; oncogenic viruslike particle; oncornavirus; Orbivirus; Orf virus; Oropouche virus; Orthohepadnavirus; Orthomyxovirus; Orthopoxvirus; Orthoreovirus; Orungo; ovine papillomavirus; ovine catarrhal fever virus; or owl monkey herpesvirus.

In some instances, the virus is Palyam virus; Papillomavirus; Papillomavirus sylvilagi; Papovavirus; parainfluenza virus; parainfluenza virus type 1; parainfluenza virus type 2; parainfluenza virus type 3; parainfluenza virus type 4; Paramyxovirus; Parapoxvirus; paravaccinia virus; Parvovirus; Parvovirus B19; parvovirus group; Pestivirus; Phlebovirus; phocine distemper virus; Picodnavirus; Picornavirus; pig cytomegalovirus-pigeonpox virus; Piry virus; Pixuna virus; pneumonia virus of mice; Pneumovirus; poliomyelitis virus; poliovirus; Polydnavirus; polyhedral virus; polyoma virus; Polyomavirus; Polyomavirus bovis; Polyomavirus cercopitheci; Polyomavirus hominis 2; Polyomavirus maccacae 1; Polyomavirus muris 1; Polyomavirus muris 2; Polyomavirus papionis 1; Polyomavirus papionis 2; Polyomavirus sylvilagi; Pongine herpesvirus 1; porcine epidemic diarrhea virus; porcine hemagglutinating encephalomyelitis virus; porcine parvovirus; porcine transmissible gastroenteritis virus; porcine type C virus; pox virus; poxvirus; poxvirus variolae; Prospect Hill virus; Provirus; pseudocowpox virus; pseudorabies virus; psittacinepox virus; or quailpox virus.

In some instances, the virus is rabbit fibroma virus; rabbit kidney vaculolating virus; rabbit papillomavirus; rabies virus; raccoon parvovirus; raccoonpox virus; Ranikhet virus; rat cytomegalovirus; rat parvovirus; rat virus; Rauscher's virus; recombinant vaccinia virus; recombinant virus; reovirus; reovirus 1; reovirus 2; reovirus 3; reptilian type C virus; respiratory infection virus; respiratory syncytial virus; respiratory virus; reticuloendotheliosis virus; Rhabdovirus; Rhabdovirus carpia; Rhadinovirus; Rhinovirus; Rhizidiovirus; Rift Valley fever virus; Riley's virus; rinderpest virus; RNA tumor virus; Ross River virus; Rotavirus; rougeole virus; Rous sarcoma virus; rubella virus; rubeola virus; Rubivirus; or Russian autumn encephalitis virus.

In some instances, the virus is SA 11 simian virus; SA2 virus; Sabia virus; Sagiyama virus; Saimirine herpesvirus 1; salivary gland virus; sandfly fever virus group; Sandjimba virus; SARS virus; SDAV (sialodacryoadenitis virus); sealpox virus; Semliki Forest Virus; Seoul virus; sheeppox virus; Shope fibroma virus; Shope papilloma virus; simian foamy virus; simian hepatitis A virus; simian human immunodeficiency virus; simian immunodeficiency virus; simian parainfluenza virus; simian T cell lymphotrophic virus; simian virus; simian virus 40; Simplexvirus; Sin Nombre virus; Sindbis virus; smallpox virus; South American hemorrhagic fever viruses; sparrowpox virus; Spumavirus; squirrel fibroma virus; squirrel monkey retrovirus; SSV 1 virus group; STLV (simian T lymphotropic virus) type I; STLV (simian T lymphotropic virus) type II; STLV (simian T lymphotropic virus) type III; stomatitis papulosa virus; submaxillary virus; suid alphaherpesvirus 1; suid herpesvirus 2; Suipoxvirus; swamp fever virus; swinepox virus; or Swiss mouse leukemia virus.

In some instances, the virus is TAC virus; Tacaribe complex virus; Tacaribe virus; Tanapox virus; Taterapox virus; Tench reovirus; Theiler's encephalomyelitis virus; Theiler's virus; Thogoto virus; Thottapalayam virus; Tick borne encephalitis virus; Tioman virus; Togavirus; Torovirus; tumor virus; Tupaia virus; turkey rhinotracheitis virus; turkeypox virus; type C retroviruses; type D oncovirus; type D retrovirus group; ulcerative disease rhabdovirus; Una virus; Uukuniemi virus group; vaccinia virus; vacuolating virus; varicella zoster virus; Varicellovirus; Varicola virus; variola major virus; variola virus; Vasin Gishu disease virus; VEE virus; Venezuelan equine encephalitis virus; Venezuelan equine encephalomyelitis virus; Venezuelan hemorrhagic fever virus; vesicular stomatitis virus; Vesiculovirus; Vilyuisk virus; viper retrovirus; viral haemorrhagic septicemia virus; Visna Maedi virus; Visna virus; volepox virus; VSV (vesicular stomatitis virus); Wallal virus; Warrego virus; wart virus; WEE virus; West Nile virus; western equine encephalitis virus; western equine encephalomyelitis virus; Whataroa virus; Winter Vomiting Virus; woodchuck hepatitis B virus; woolly monkey sarcoma virus; wound tumor virus; WRSV virus; Yaba monkey tumor virus; Yaba virus; Yatapoxvirus; yellow fever virus; or the Yug Bogdanovac virus.

Methods described herein may be used to detect pathogens such as fungi, molds, or parasites. In some instances, the fungi is Blastomyces, Coccidiomycosis, Cryptococcus gattii, Cryptococcus neoformans, paracoccidioidomycosis, hisoplasmosis, Candida (e.g., Candida auris), aspergillosis spp. (e.g., fumigatus, flavus), Pneumocystis jirovecii, mucormycetes, or taloromyces. In some instances, methods described herein are used to detect parasites. In some instances, pathogens include but are not limited to Toxoplasma gondii, Trypanosoma cruzi, Cryptosporidium parvum, Encephalitozoon spp., or Stachybotrys chartarum.

Methods described herein may be used to detect bacteria, such as pathogenic bacteria. In some instances, the bacteria is S. aureus, S. epidermitis, Helicobacter pylori, Enterococcus faecalis, or Enterococcus faecium. In some instances, bacteria include multi-drug resistant bacteria.

Methods described herein may detect low concentrations or amounts of viruses in a sample. In some instances, the amount of a virus is represented in terms of the number of genome copies (cp). In some instance, a method described herein detects about 1, 2, 5, 10, 15, 20, 25, 50, 100, 200, 500, 1000, 5000, 10,000, 50,000, 100,000, 500,000, or about 1,000,000 cp of a virus in a sample. In some instance, a method described herein detects no more than 1, 2, 5, 10, 15, 20, 25, 50, 100, 200, 500, 1000, 5000, 10,000, 50,000, 100,000, 500,000, or no more than 1,000,000 cp of a virus in a sample. In some instance, a method described herein detects at least 1, 2, 5, 10, 15, 20, 25, 50, 100, 200, 500, 1000, 5000, 10,000, 50,000, 100,000, 500,000, or at least 1,000,000 cp of a virus in a sample. In some instance, a method described herein detects 1-10, 1-100, 1-500, 1-1000, 1-5000, 1-10,000, 10-1000, 10-5000, 10-100,000, 100-10,000, 100-100,000, 100-1,000,000, 1000-5000, 1000-10,000, 1000-50,000, or 50,000-1,000,000 cp of a virus in a sample.

Described herein are methods of sample analysis comprising analysis of RNA and DNA from a sample source comprising a putative virus. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library. In some instances, a pull-down purification step is not required.

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

RT reactions may be used to reverse transcribe RNA (e.g., viral RNA). Various reaction conditions and mixes are in some instances used for generating cDNA libraries for transcriptome analysis of virus-containing samples, wherein the cDNA libraries are analyzed by methods such as LAMP or PTA. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length). In some instances an RT reaction mix contains gelatin or bovine serum albumin.

Primary Template-Directed Amplification

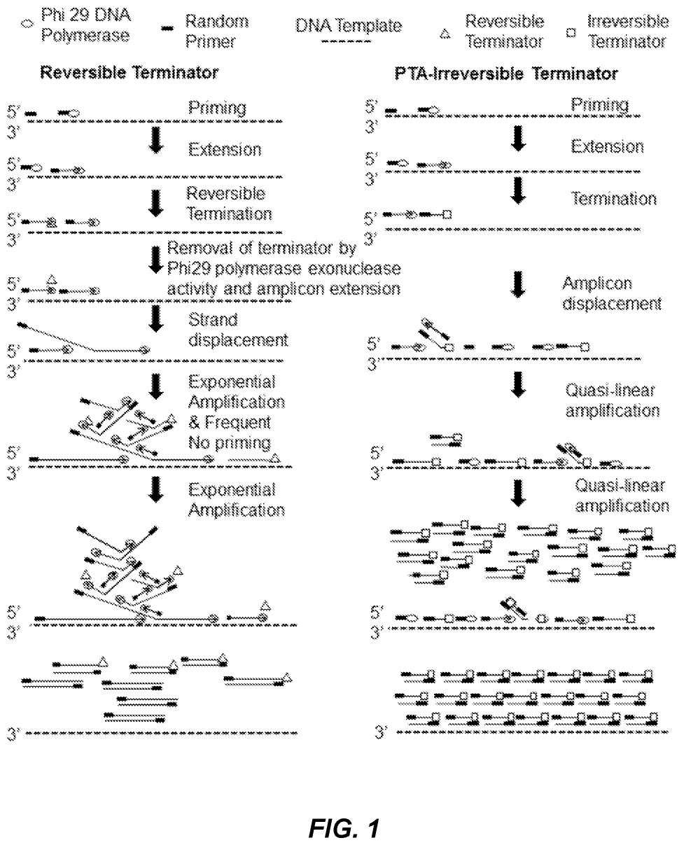

Described herein are nucleic acid amplification methods, such as “Primary Template-Directed Amplification (PTA).” Such methods in some instances are combined with reverse transcription. In some instance, PTA is used to detect low amounts of nucleic acids, such as viral cDNA or other nucleic acids. With the PTA method, amplicons are preferentially generated from the primary template (“direct copies”) using a polymerase (e.g., a strand displacing polymerase) (FIG. 1). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions. In some instances, terminator removal is not required prior to amplification and/or adapter ligation.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Φ29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(−) Bst; Aliotta et al., Genet. Anal. (Netherlands) 12:185-195 (1996)), exo(−)Bca DNA polymerase (Walker and Linn, Clinical Chemistry 42:1604-1608 (1996)), Bsu DNA polymerase, Vent_{R} DNA polymerase including Vent_{R} (exo-) DNA polymerase (Kong et al., J. Biol. Chem. 268:1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351).

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances, a terminator pairing with cytosine is present at about 3%, 5%, 10%, 15%, 20%, 25%, or 50% higher concentration than the average terminator concentration. In some instances, a terminator pairing with thymine is present at about 3%, 5%, 10%, 15%, 20%, 25%, or 50% higher concentration than the average terminator concentration. In some instances, a terminator pairing with guanine is present at about 3%, 5%, 10%, 15%, 20%, 25%, or 50% higher concentration than the average terminator concentration. In some instances, a terminator pairing with adenine is present at about 3%, 5%, 10%, 15%, 20%, 25%, or 50% higher concentration than the average terminator concentration. In some instances, a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and Vent_{R} (exo-).

Primers and Amplicon Libraries

Described herein are amplicon libraries resulting from amplification of at least one target nucleic acid molecule (e.g., viral nucleic acid). Such libraries are in some instances generated using the methods described herein, such as those using terminators. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of amplification cycles (PCT or isothermal amplification). In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further PCR amplification. In some instances, such additional steps precede a sequencing step. In some instances, no more than 70, 60, 50, 40, 30, 25, 20, 15, 13, 11, 10, 9, 8, 7, 6, 5, 4, or 3 cycles are used to generate copies of the target nucleic acid molecule. In some instances, about 70, 60, 50, 40, 30, 25, 20, 15, 13, 11, 10, 9, 8, 7, 6, 5, 4, or about 3 cycles are used to generate copies of the target nucleic acid molecule. In some instances, about 3, 4, 5, 6, 7, 8, 10, 15, 20, 25, 30, 35, 40, 50, 60, or about 70 cycles are used to generate copies of the target nucleic acid molecule. In some instances, 2-4, 2-5, 2-7, 2-8, 2-10, 2-15, 3-5, 3-10, 3-15, 4-10, 4-15, 5-10, 5-15, 5-70, 10-70, 20-70, 30-70, 40-70, 50-70, 10-20, 10-30, or 10-40 cycles are used to generate copies of the target nucleic acid molecule. Amplification using PTA in some instances is combined with reverse transcription (RT-PTA).

Amplicon libraries of polynucleotides generated from the PTA methods and compositions (terminators, polymerases, etc.) described herein in some instances have increased uniformity. Uniformity, in some instances, is described using a Lorenz curve or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example no more than 100 million, 200 million, 300 million, 400 million, or no more than 500 million reads are obtained. In some instances, the read length is about 50,75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process. In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential amplification of smaller fragments during PCR while preparing sequencing libraries, as well as a result of the preferential formation of clusters from smaller sequencing library fragments during Illumina sequencing. Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, purified nucleic acid from individual cells are contacted with one or more beads having a unique set of sample barcodes and/or UMI sequences in order to identify the purified nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, purified nucleic acid from individual cells are contacted with a droplet having a unique set of sample barcodes and/or UMI sequences in order to identify the purified nucleic acid from the individual cell. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, Calif., USA, and Illumina, San Diego, Calif., USA)); Chromium (10× Genomics, Pleasanton, Calif., USA)); Rhapsody Single-Cell Analysis System (BD, Franklin Lakes, N.J., USA); Tapestri Platform (MissionBio, San Francisco, Calif., USA)), Nadia Innovate (Dolomite Bio, Royston, UK); C1 and Polaris (Fluidigm, South San Francisco, Calif., USA); ICELL8 Single-Cell System (Takara); MSND (Wafergen); Puncher platform (Vycap); CellRaft AIR System (CellMicrosystems); DEPArray NxT and DEPArray System (Menarini Silicon Biosystems); AVISO CellCelector (ALS); and InDrop System (1CellBio).

PTA primers may comprise a sequence-specific or random primer, an address tag, a cell barcode and/or a unique molecular identifier (UMI). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 10¹⁰ unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors. In some instances, primers comprise reporter and/or quenching moieties which allow detection of amplicons.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods (including those which produce detectable, real-time signals) which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLiD, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem. 47:164-172). In some instances, the amplified nucleic acid molecules are shotgun sequenced.

### Kits

Described herein are kits for the detection of nucleic acids from samples. In some instances, a kit described herein comprises one or more of a sampling device, one or more positive control nucleic acids, negative control, primers, probes, reverse transcriptase, polymerase, sample plates, sample tubes, pipets, or lysis buffer. In some instances, a lysis buffer comprises a reducing agent. In some instances, a lysis buffer comprises proteinase K or proteinase pk. In some instances, a kit described herein comprises an qRT-PCR master mix. In some instances, a master mix comprises a polymerase (e.g., TaqMan, or other polymerase), uracil-N-glycosylase, dNTPs with dUTP, passive reference dyes (e.g., ROX dye), and other buffers. In some instances, the plate is a 96 or 386 well plate. In some instances, the primers and probes are configured to detect a virus (e.g., Covid-19, SARS, or MERS). In some instances, the master mix is attached to a bead. In some instances, kits further comprise reagents for RT-LAMP or RT-PTA methods.

Described herein are kits facilitating the practice of the PTA method with RT-PCR to detect nucleic acids. Various combinations of the components set forth above in regard to exemplary reaction mixtures and reaction methods can be provided in a kit form. A kit may include individual components that are separated from each other, for example, being carried in separate vessels or packages. A kit in some instances includes one or more sub-combinations of the components set forth herein, the one or more sub-combinations being separated from other components of the kit. The sub-combinations in some instances are combinable to create a reaction mixture set forth herein (or combined to perform a reaction set forth herein). In particular embodiments, a sub-combination of components that is present in an individual vessel or package is insufficient to perform a reaction set forth herein. However, the kit as a whole in some instances includes a collection of vessels or packages the contents of which can be combined to perform a reaction set forth herein.

A kit can include a suitable packaging material to house the contents of the kit. The packaging material in some instances is constructed by well-known methods, preferably to provide a sterile, contaminant-free environment. The packaging materials employed herein include, for example, those customarily utilized in commercial kits sold for use with nucleic acid sequencing systems. Exemplary packaging materials include, without limitation, glass, plastic, paper, foil, and the like, capable of holding within fixed limits a component set forth herein. The packaging material can include a label which indicates a particular use for the components. The use for the kit that is indicated by the label in some in instances is one or more of the methods set forth herein as appropriate for the particular combination of components present in the kit. For example, a label in some instances indicates that the kit is useful for a method of detecting mutations in a nucleic acid sample using the PTA method. Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like. It will be understood that not all components necessary for a particular reaction need be present in a particular kit. Rather one or more additional components in some instances are provided from other sources. The instructions provided with a kit in some instances identify the additional component(s) that are to be provided and where they can be obtained. In one embodiment, a kit provides at least one amplification primer; at least one nucleic acid polymerase; a mixture of at least two nucleotides, wherein the mixture of nucleotides comprises at least one terminator nucleotide which terminates nucleic acid replication by the polymerase; and instructions for use of the kit. In some instances, the kit provides reagents to perform the methods described herein, such as PTA. In some instances, a kit further comprises reagents configured for gene editing (e.g., Crispr/cas9 or other method described herein). In some instances, a kit comprises a variant polymerase described herein.

In a related aspect, the invention provides a kit comprising a reverse transcriptase, a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In one embodiment of the kits of the invention, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ hexanediol spacer nucleotides, acyclonucleotides, and combinations thereof.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1: Primary Template-Directed Amplification of Single Cells

Single Cell Capture by FACS Sorting. A low bind 96-well PCR plate was placed on a PCR cooler. 3 μL of Cell Buffer was added to all the wells where cells will be sorted. The plate was sealed with a sealing film and kept it on ice until ready to use. After single cell sorting, the plate is sealed. The plate was mixed for 10 seconds at 1400 RPM on a PCR Plate Thermal Mixer at room temperature, spun briefly, and placed on ice. Alternatively, plates containing sorted cells were stored on dry ice with a seal or at −80° C. until ready.

Single Cell Whole Genome Amplification with PTA (FIG. 1). After adding reagents to plates containing cells, an RPM controlled mixer was used PCR cooler at set to −20° C. for 2 hrs and thawed for 10 min or alternatively the following reactions were conducted on ice. Reactions were assembled in a DNA-free pre-PCR hood. All reagents were thawed on ice until ready to use. Reagents collectively add randomized primers of 8-15 nt in length, dNTPs, Phi29 polymerase, buffer, and further contain 10% alpha-thio-ddNTPs relative to dNTPs. Before use, each reagent was vortexed for 10 sec and spun briefly. Reagents were dispensed to the wall of the tube without touching cell suspension. 96-well PCR plate containing cells were placed on the PCR cooler. If cells were stored at −80° C., cells were thawed on ice for 5 minutes, spun for 10 seconds, then the plate placed on the PCR cooler (or ice). 1× Reagent Mix was prepared by diluting 12× mix, mixing on the vortexer, and briefly spinning tube. MS Mix was prepared by combining 1× reagent mix and lysis buffer, mixing on the vortexer, and briefly spinning tube. 3 L of MS Mix was added to each well of the plate, and the plate was sealed with the sealing film. After spinning for 10 sec, mixing at room temperature for 1 min at 1400 rpm (plate mixer), and spinning for 10 sec, plate was placed back on PCR cooler (or ice) for 10 minutes. 3 μL of neutralization buffer, was then added, and the plate was sealed with the plate film. After spinning for 10 sec, mixing at room temperature for 1 min at 1400 rpm (plate mixer), spinning for 10 sec, the plate was placed back on the PCR cooler. 3 μL of buffer was added, and the plate sealed with the plate film. Next, the plate was spun for 10 sec, mixed at room temperature for 1 min at 1400 rpm (plate mixer), and spun for 10 sec followed by incubating at room temperature for 10 min. During the incubation step, the Reaction Mix was prepared by combining the components in the order (nucleotide/terminator reagents, 5.0 μL; 1× reagent mix, 1.0 μL; Phi29 polymerase (4×), 0.8 μL; singe-stranded binding protein reagent, 1.2 μL), followed by mixing gently and thoroughly by pipetting up and down 10 times, then spun briefly. When the incubation is completed, the plate is placed on the PCR cooler (or ice). 8 μL of Reaction Mix was added to each sample while the plate is still on the PCR cooler (or ice), and mixed at room temperature for 1 min at 1000 rpm in plate mixer, then spun briefly. The plate is placed on a thermal cycler (lid set to 70° C.) with the following program: 30° C. for 10 hrs, 65° C. for 3 min, 4° C. hold.

Amplified DNA Cleanup. Capture beads were allowed to equilibrate to room temperature for 30 min. Beads are mixed thoroughly, and then 40 μL of beads were added to each reaction well (vortex and spin). Beads were aspirated prior to each dispensing step, incubated at room temperature for 10 minutes, and the sample plate briefly centrifuged. The plate was placed on a magnet for 3 minutes or until the supernatant cleared. While on the magnet, the supernatant is removed and discarded, being careful not to disturb the beads containing DNA. While on magnet, 200 μL of freshly prepared 80% ethanol was added to the beads and incubated for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 40 μL of elution buffer, incubated for 2 minutes at room temperature, and placed on the magnet for 3 minutes, or until the supernatant clears. 38 μL of the eluted DNA was transferred to a new plate, for DNA quantification. DNA was then ready to use in downstream applications such as PCR or Real Time PCR.

DNA Quantification. Quantitate DNA using the High Sensitivity dsDNA Assay kit (Qubit) as per manufacturer. Size fragment analysis was completed to ensure proper amplification product size. Fragment size distribution was determined by running 1 μL of PTA product on an E-Gel EX, or 1 μL of 2 ng/L in a High Sensitivity Bioanalyzer DNA Chip.

End Repair and A-tailing. 500 ng of amplified DNA was added to a PCR tube. DNA volume was adjusted to 35 μL with RT-PCR grade water. The End-Repair A-Tail Reaction was assembled on a PCR cooler (or ice) as follows: Amplified DNA (500 ng total DNA/Rxn, 35 μL), RT-PCR grade water (10 μL), fragmentation buffer (5 μL), ER/AT buffer (7 μL), ER/AT enzyme (3 μL) to a total volume of 60 μL, which was mixed thoroughly and spun briefly. The mixture was then incubated at 65° C. on a thermal cycler with the lid at 105° C. for 30 minutes.

Adapter Ligation. Multi-Use Library Adapters stock plate was diluted to 1× by adding 54 μL of 10 mM Tris-HCl, 0.1 mM EDTA, pH 8.0 to each well. In the same plate/tube(s) in which end-repair and A-tailing was performed, each Adapter Ligation Reaction was assembled as follows: ER/AT DNA (60 μL), 1× Multi-Use Library Adapters (5 μL), RT-PCR grade water (5 μL), ligation buffer (30 μL), and DNA ligase (10 μL) to a total volume of 110 μL. After thorough mixing and brief spin, the mixture is incubated at 20° C. on thermal cycler for 15 minutes (heated lid not required).

Post Ligation Cleanup. Beads were allowed to equilibrate to room temperature for 30 minutes then mixed thoroughly and immediately before pipetting. In the same plate/tube(s), a 0.8×SPRI cleanup was assembled as follows: adapter-ligated DNA (110 μL), and beads (88 μL) to a final volume of 198 μL. The mixture is mixed thoroughly and incubated for 10 min at room temperature, and the plate/tube(s) are placed on the magnet for 2 minutes, or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 20 μL of elution buffer, incubated for 2 minutes at room temperature, and placed on the magnet for 3 minutes, or until the supernatant clears.

Library Amplification. In the same plate/tube(s) containing the DNA-Bead slurry, each library amplification reaction is assembled as follows: adapter ligated library (20 L), 10×KAPA library amplification primer mix (5 μL), and 2×KAPA HiFi Hotstart ready mix (25 μL) to a total volume of 50 μL. After mixing thoroughly and spinning briefly, amplification is conducted using the cycling protocol: Initial Denaturation 98° C. @ 45 sec (1 cycle), Denaturation 98° C. @ 15 sec; Annealing 60° C. 30 sec; and Extension 72° C. 30 sec (10 cycles), Final Extension 72° C. @ 1 min for 1 cycle, and HOLD 4° C. indefinitely. The heated lid was set to 105° C. The plate/tube(s) were stored at 4° C. for up to 72 hours, or directly used for Post-Amplification Cleanup.

Post Amplification Clean up. Beads were allowed to equilibrate to room temperature for 30 minutes. Beads thoroughly and immediately before pipetting, and in the same plate/tube(s), a 0.55×SPRI cleanup was assembled as follows: amplified library (50.0 μL) and beads (27.5 μL) to a total volume of 77.5 μL, followed by thorough mixing and incubation for 10 min at room temperature. Plate/tube(s) were placed on the magnet for 3 minutes, or until the supernatant clears. While on the magnet, the supernatant was transferred to a new plate/tube(s) being careful not to transfer any beads.

In a plate/tube(s), a 0.25×SPRI cleanup was assembled as follows: 0.55× Cleanup Supernatant (77.5 μL), and beads (12.5 μL) to a total volume of 90.0 μL. After thorough mixing, the mixture was spun down and incubated for 10 min at room temperature. Plate/tube(s) were placed on the magnet for 3 minutes or until the supernatant clears. While on the magnet, the supernatant was removed and discarded being careful not to disturb any beads, followed by washing with 200 μL of freshly prepared 80% ethanol to the beads and incubating for 30 seconds at room temperature. While still on the magnet, the first ethanol wash is removed and discarded, taking care not to disturb the beads. Another 200 μL of freshly prepared 80% ethanol is added to the beads, and then incubated for 30 seconds at room temperature. The second ethanol wash is then removed and discarded, taking care not to disturb the beads. Any remaining ethanol from the wells is discarded. The beads are then incubated at room temperature for 5 minutes to air-dry beads, then the plate was removed from the magnet. Beads were then re-suspended in 42 μL of elution buffer, incubated for 2 minutes at room temperature, and placed on the magnet for 3 minutes, or until the supernatant clears. 40 μL of the eluted DNA was transferred to a new plate, for DNA quantification.

Library Quantification. The amplified library is quantitated using a Qubit dsDNA kit as per manufacturer. Fragment size distribution was determined by running 1 μL of library on an E-Gel EX, or 1 μL of 2 ng/L in a Bioanalyzer DNA Chip.

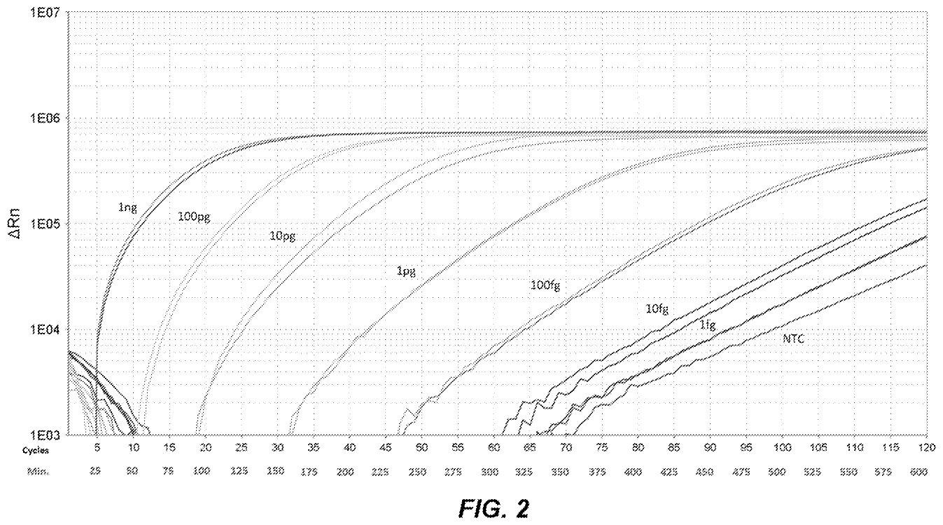

Use of NA12878 DNA in various concentrations as a control demonstrates the PTA reaction has a significant dynamic assay range (6-7 logs, FIG. 2). Single cells may be amplified in 2-3 hours.

### Example 2: Bacterial Detection Assay from Buccal Swab

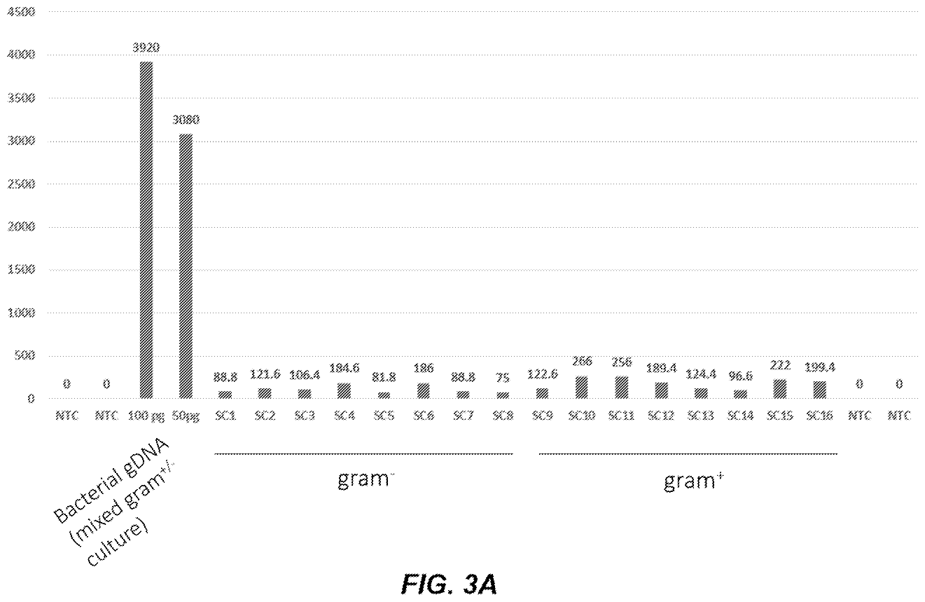

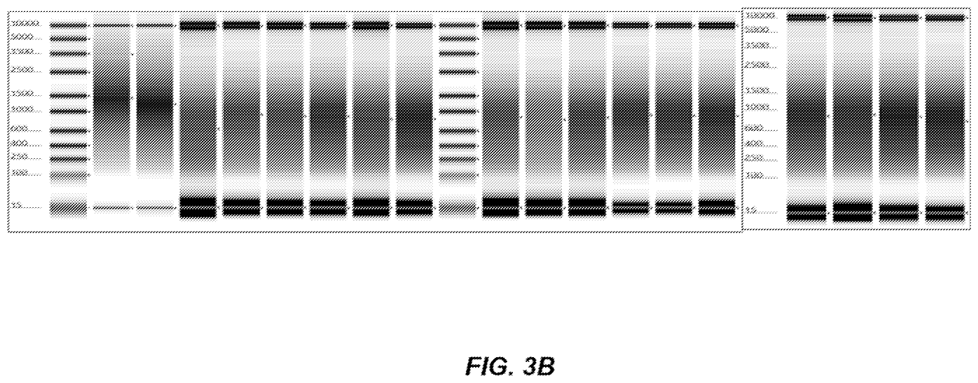

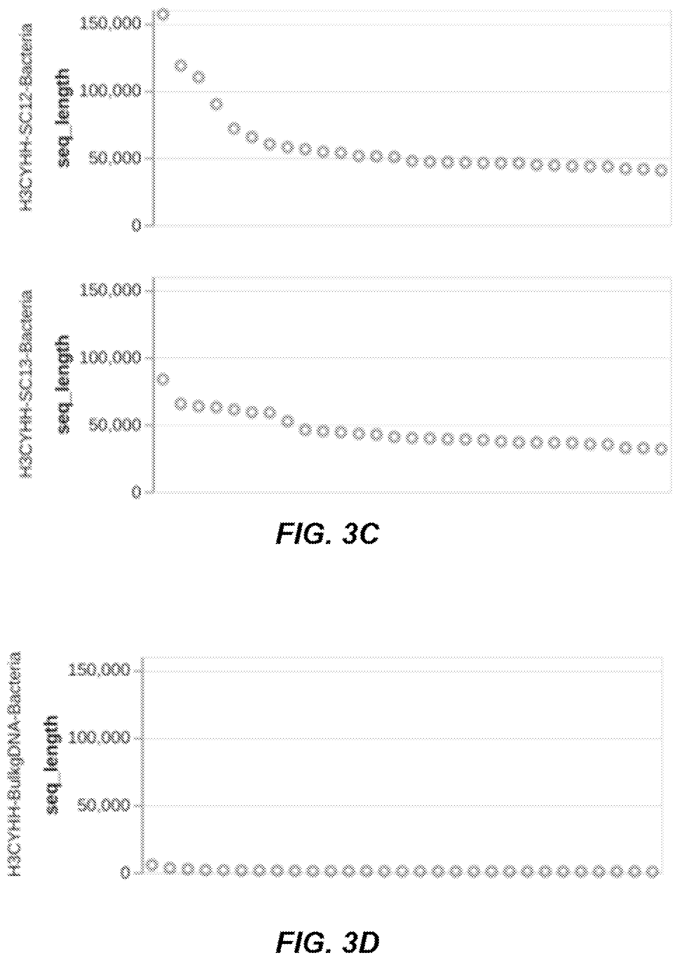

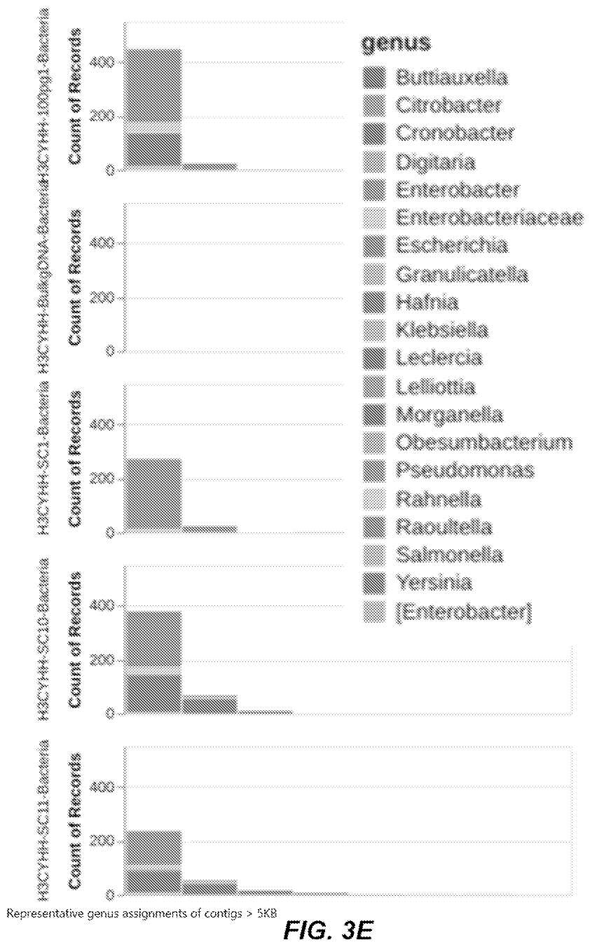

Bacteria was analyzed using the general methods of Example 1. First, a buccal swabs were obtained from a subject and cultured from a glycerol stock, pre-cultured in media at 37° C. for two nights, filtered (20 μm), and washed twice with PBS to produce a bacterial suspension. The sample was then divided into analysis and control groups, wherein the control group was treated with 2-proponol for 30 minutes to kill bacteria in the culture. Cells were tagged and sorted using Syto9 (gram+, gram−), Hexidium iodide (gram+), and Propidium iodide (dead) into a 96 well plate, treated with lysozyme, incubated for 30 minutes at room temperature, and subjected to five freeze-thaw cycles to release nucleic acids. These nucleic acids where then amplified using the PTA method (randomized primers of 8-15 nt in length, dNTPs, Phi29 polymerase, buffer, and further contain 10% alpha-thio-ddNTPs relative to dNTPs), amplified, ligated to adapters to generate a library, and the library sequenced. PTA sequencing results are shown in FIGS. 3A-3B. Sequencing data was assembled into genomes/contigs using SPADES. “Best-match” taxa was assigned to each contig using BLASTN. Many large contigs were assembled (>100kb, 2.5% of the E. coli genome) (FIG. 3C). The bulk sample comprised very small contigs (FIG. 3D). Samples all mapped predominantly to two genera of Enterobacteriaceae; both were Gram-negative (FIG. 3E).

### Example 3: Detection of Gram Negative Bacteria

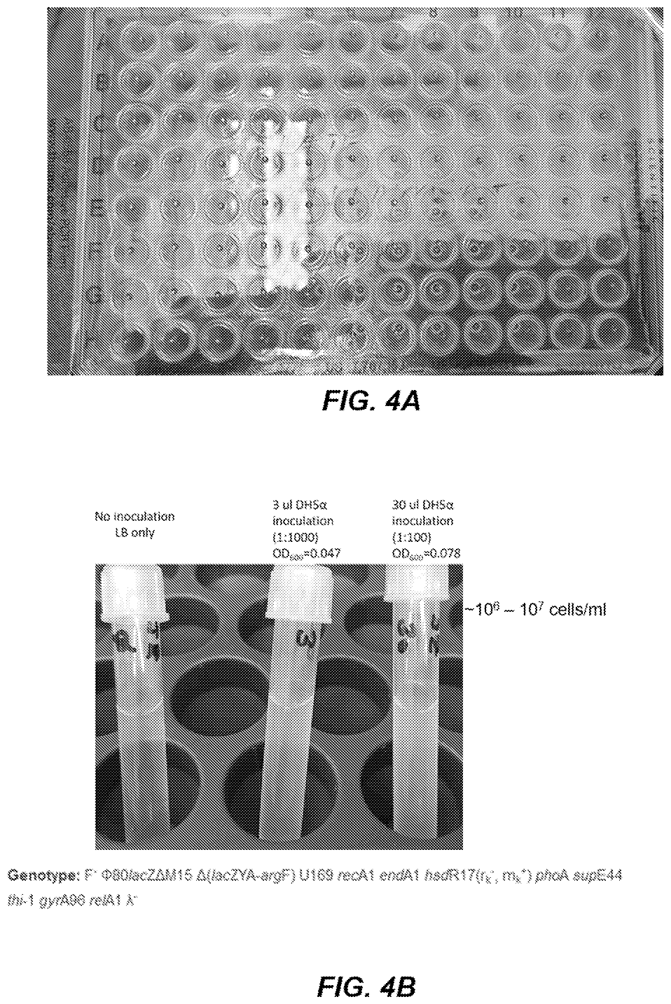

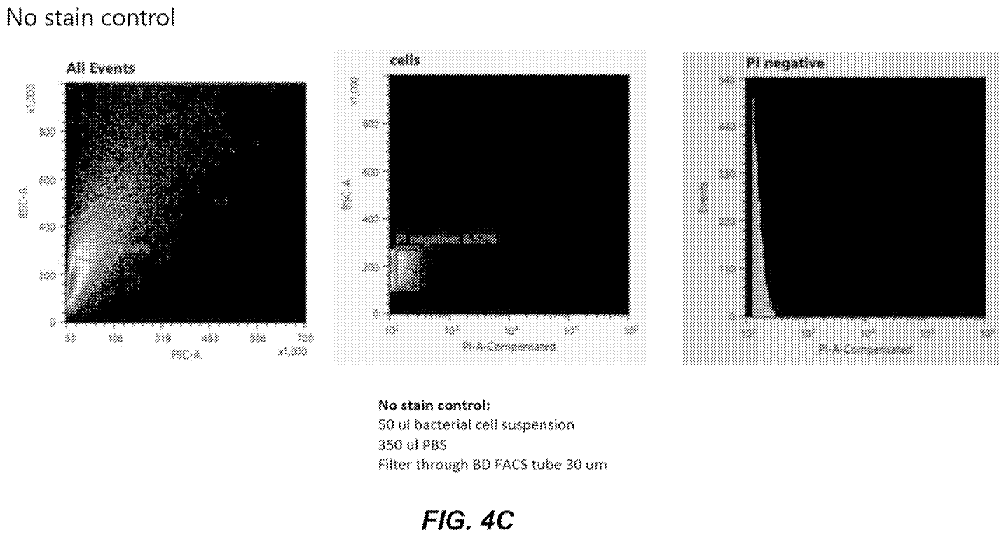

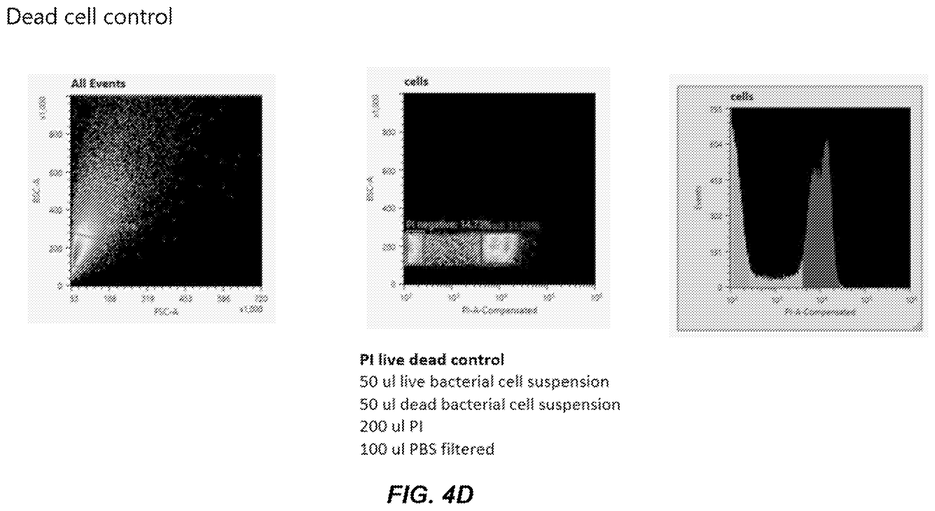

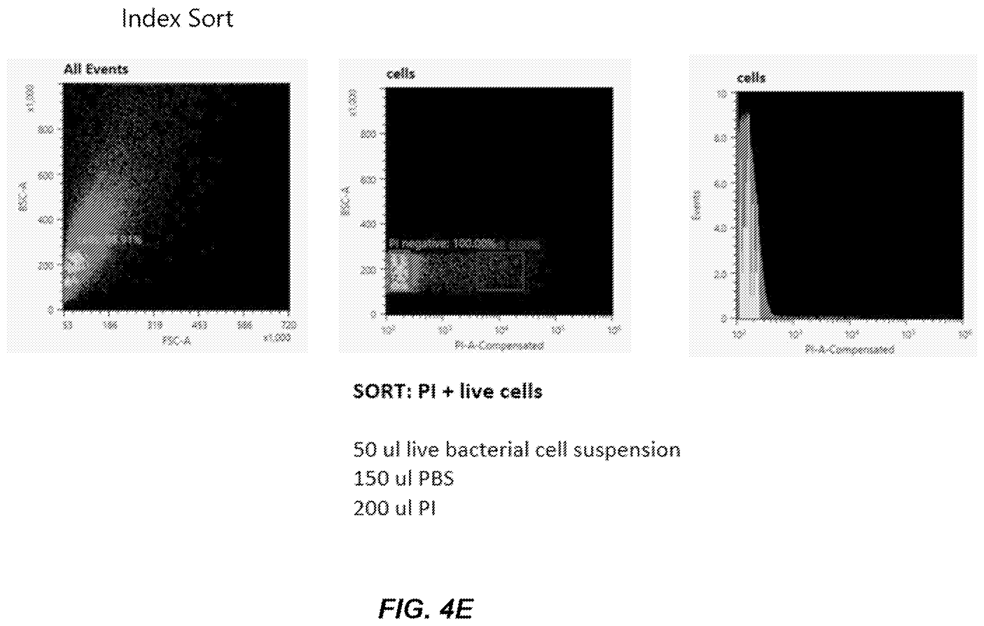

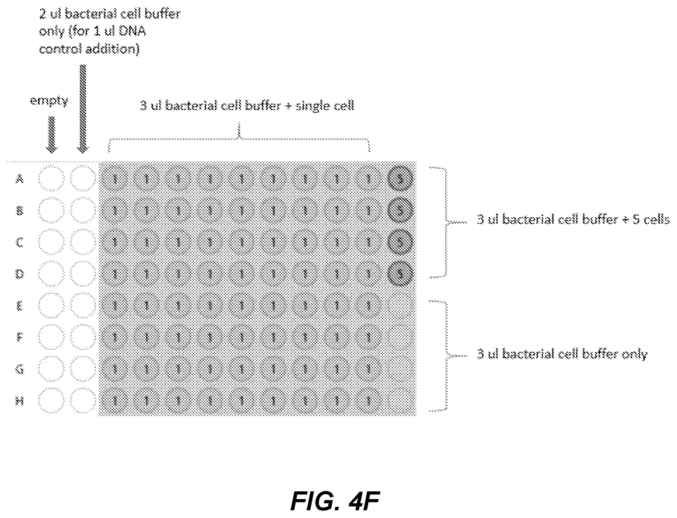

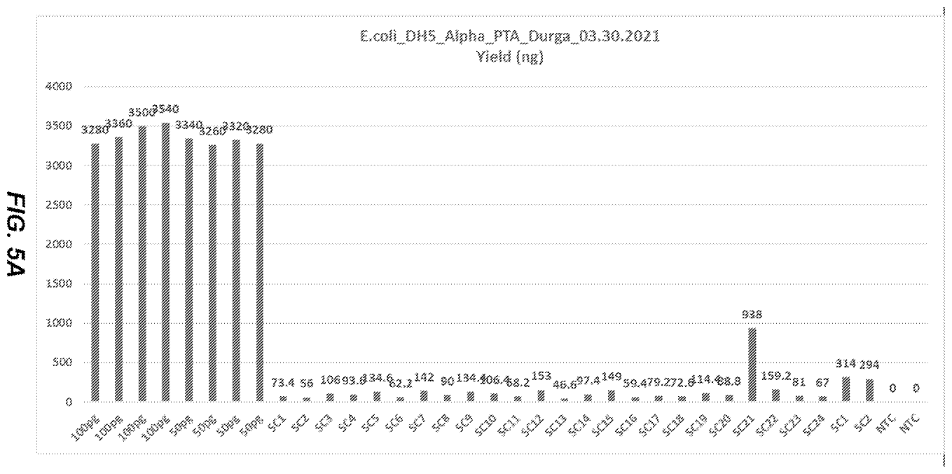

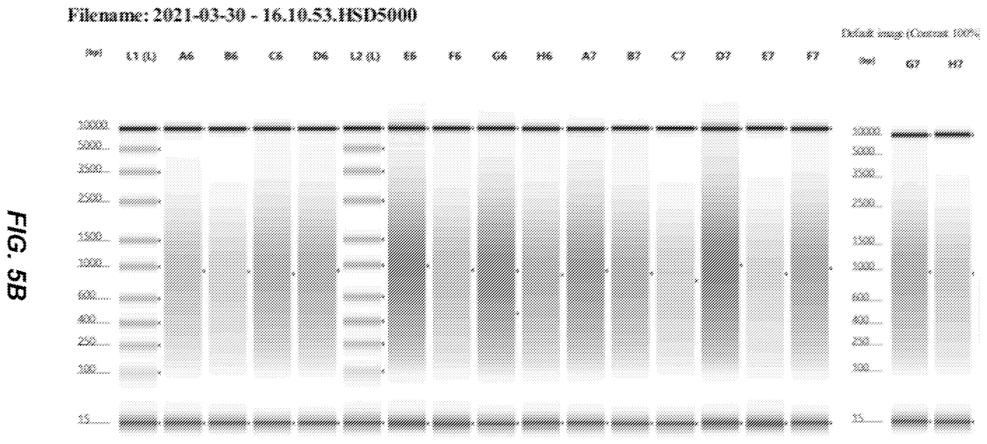

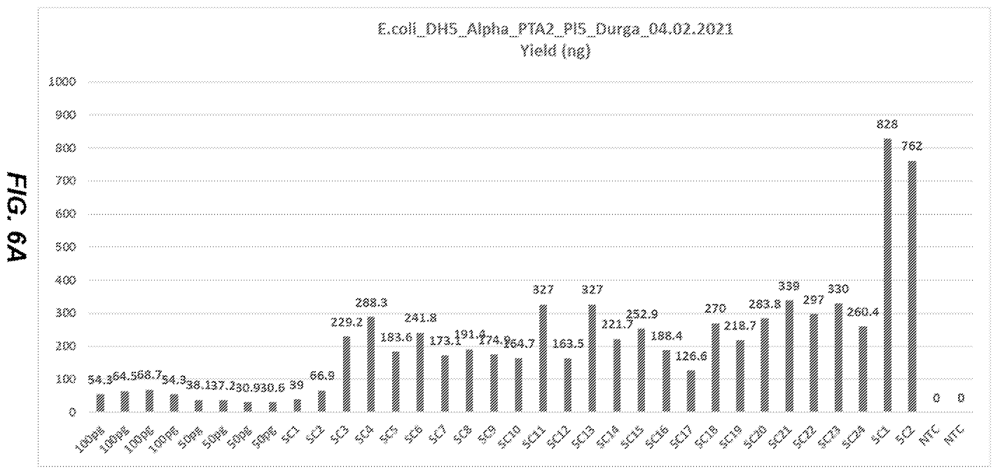

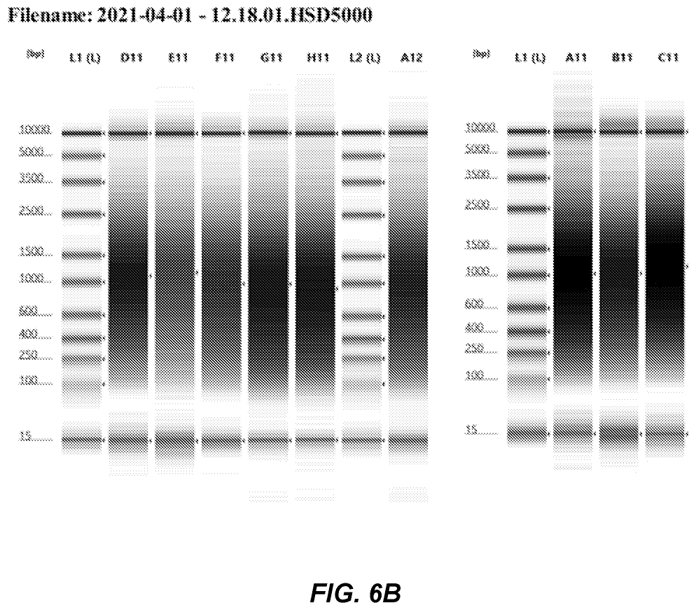

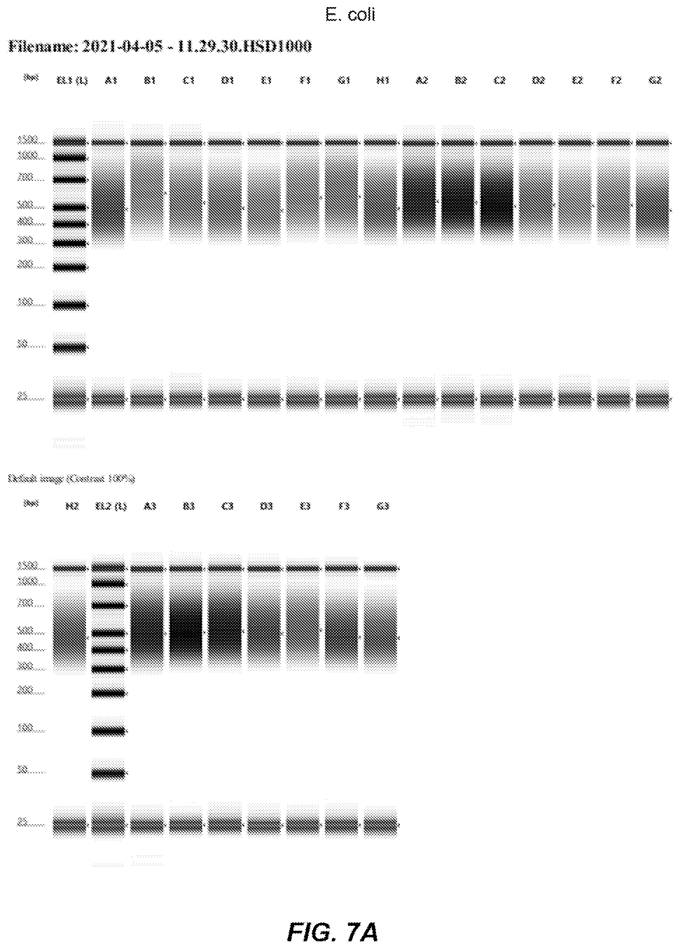

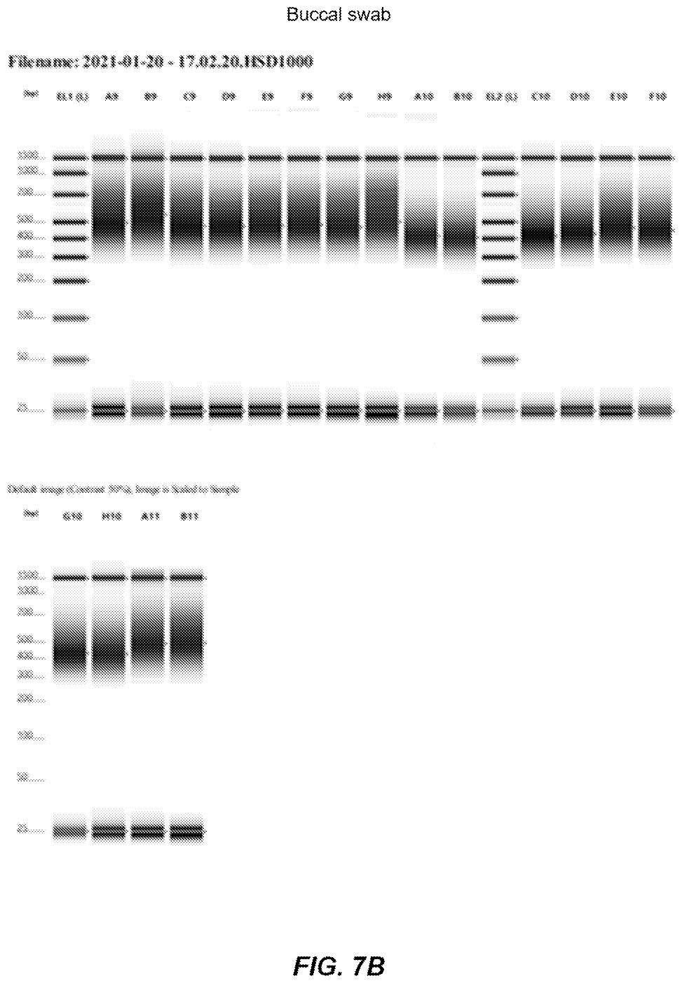

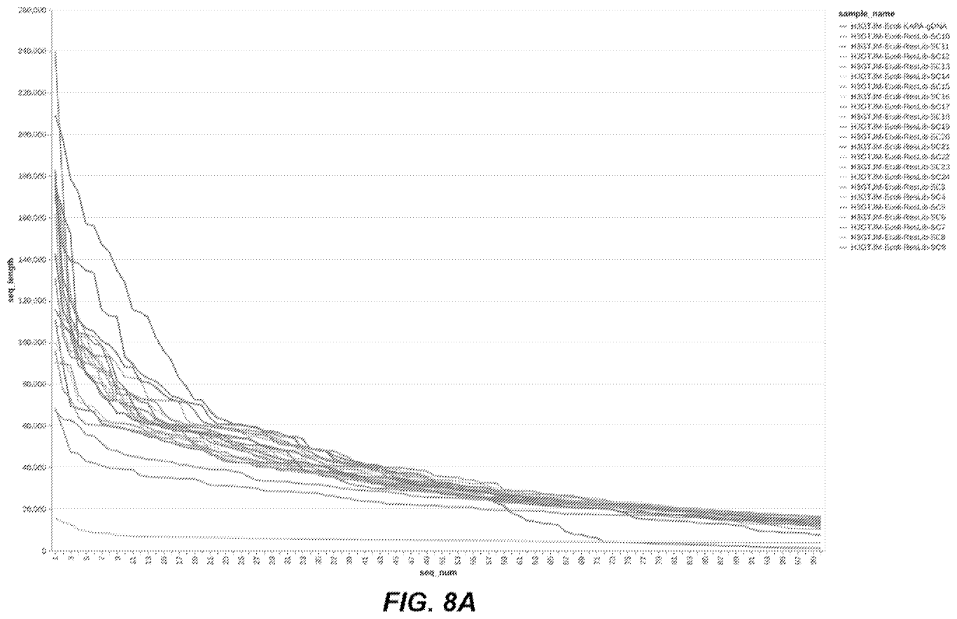

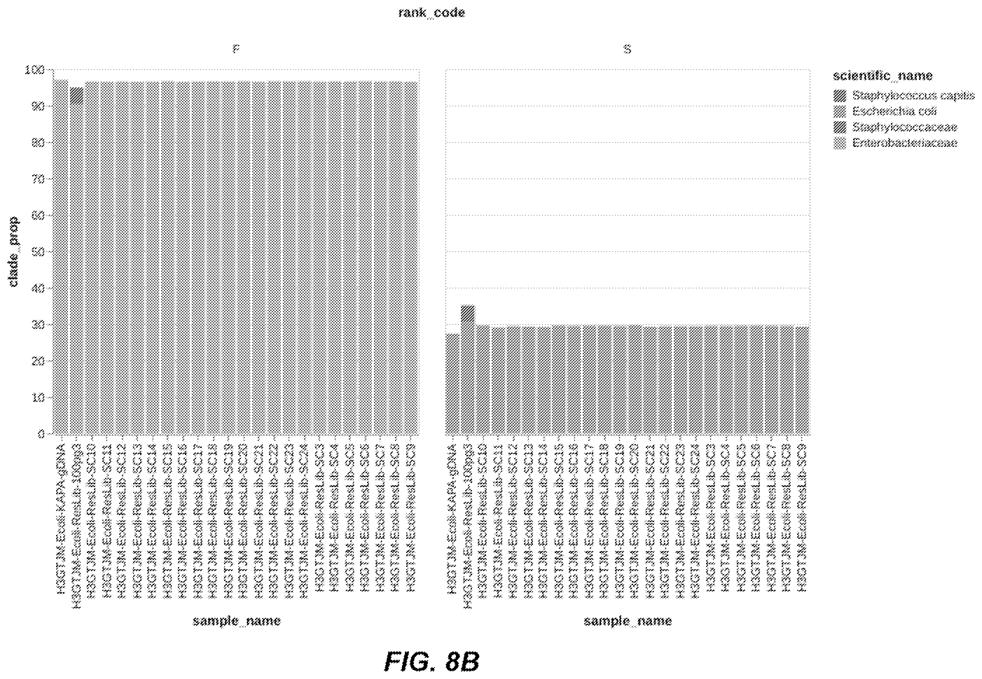

Following the general methods of Examples 1 and 2, DH5α E. coli (genotype: F-Φ80lacZΔM15 Δ(lacZYA-argF) U169 recA1 endA hsdR17(rₖ⁻, mₖ⁺)phoA supE44) were cultured (FIGS. 4A-4B), stained, sorted by FACS (FIGS. 4C-4E) in a plate. Sorting conditions included empty wells (control, column 1), 2 microliters bacterial cell buffer only (column 2, for 1 microliter DNA control addition), 3 microliters bacterial cell buffer+a single cell (columns 3-11), 3 microliters bacterial cell buffer+5 cells (column 12, rows A-D), and microliters bacterial cell buffer only (column 12, rows E-H) (FIG. 4F). Gain settings for sorting were: Laser: 488 nm, 638 nm; Sample Pressure: 4; AD advanced settings: forward window extension: 50, back window extension: 50; sensor gain: FSC: 16, BSC: 48%, FL1: 40%, FL2: 40%, FL3: 46.5%, FL4: 40%, FL5: 40%, FL6: 40%. After amplification with the PTA reaction, yields and amplicon sizes were examined from two replicates (FIGS. 5A-6B). DNA libraries were then constructed, analyzed by gel electrophoresis, and sequenced (FIGS. 7A-7B). Contig assembly is shown in FIG. 8A. Most samples had contigs ˜ 100 KB in length, and the gDNA sample had the largest contigs. Contig identification is shown in FIG. 8B. Most samples had nearly identical taxon distribution, and mapped highest to E. coli at the family level. The 100 pg control had a small proportion of Staphylococcaceae. Read taxon assignments are show in Table 1. 90.37% of reads mapped to Enterobacteriaceae, and 30.63% mapped to Escherichia coli.

TABLE 1
Representative Taxon Assignments

| | | | | | |
|---|---|---|---|---|---|
| 2.51 | 76347 | 76347 | U | 0 | unclassified |
| 97.49 | 2962009 | 24649 | R | 1 | root |
| 96.67 | 2937256 | 1324 | R1 | 131567 | cellular organisms |
| 96.59 | 2934648 | 5337 | D | 2 | Bacteria |
| 91.31 | 2774197 | 5193 | P | 1224 | Proteobacteria |
| 91.13 | 2768724 | 4969 | C | 1236 | Gammaproteobacteria |
| 90.93 | 2762842 | 15465 | O | 91347 | Enterobacterales |
| 90.37 | 2745707 | 1723250 | F | 543 | Enterobacteriaceae |
| 32.93 | 1000626 | 58988 | G | 561 | Escherichia |
| 30.63 | 930700 | 916477 | S | 562 | Escherichia coli |
| 0.07 | 2182 | 320 | S1 | 83333 | Escherichia coli K-12 |
| 0.05 | 1400 | 151 | S2 | 511145 | Escherichia coli str. K-12 substr. MG1655 |
| 0.04 | 1249 | 1249 | S3 | 879462 | Escherichia coli str. K-12 substr. MG1655star |
| 0.01 | 366 | 366 | S2 | 1110693 | Escherichia coli str. K-12 substr. MDS42 |
| 0 | 94 | 94 | S2 | 316407 | Escherichia coli str. K-12 substr. W3110 |
| 0 | 1 | 1 | S2 | 1245474 | Escherichia coli ER2796 |
| 0 | 1 | 1 | S2 | 1403831 | Escherichia coli str. K-12 substr. MC4100 |

### Example 4: Detection of Gram Positive Bacteria

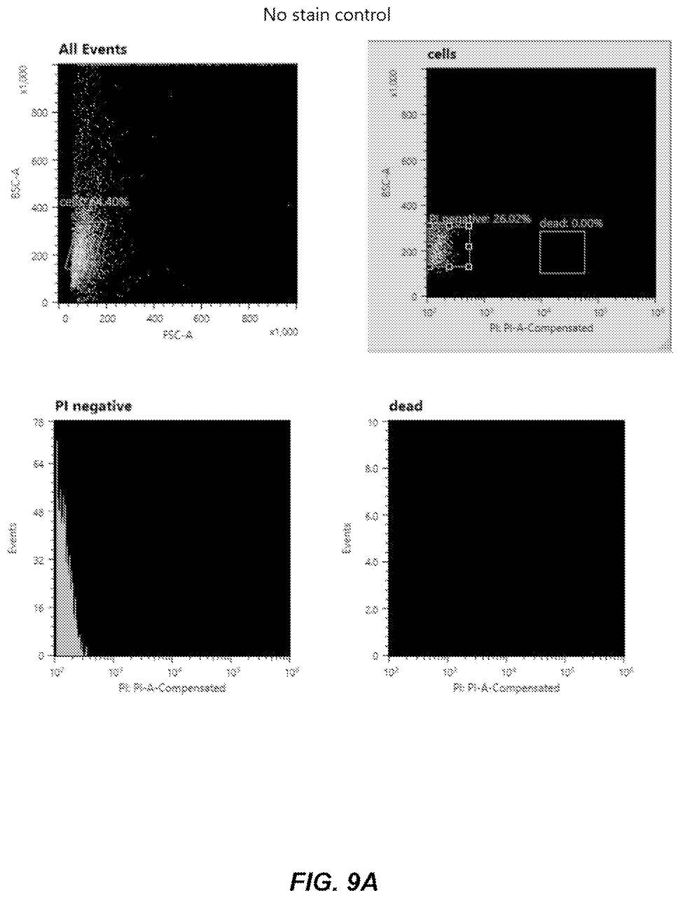

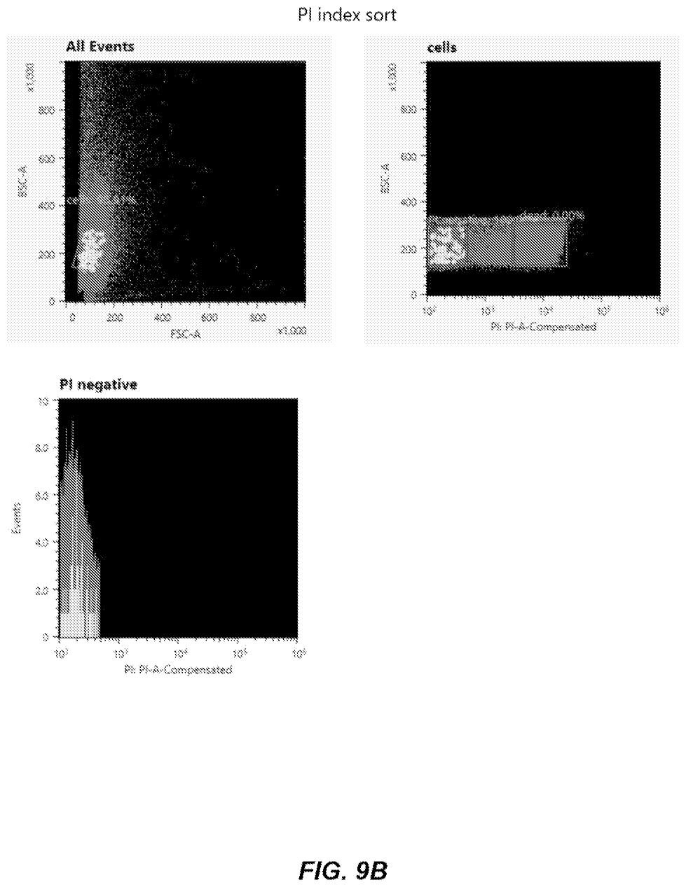

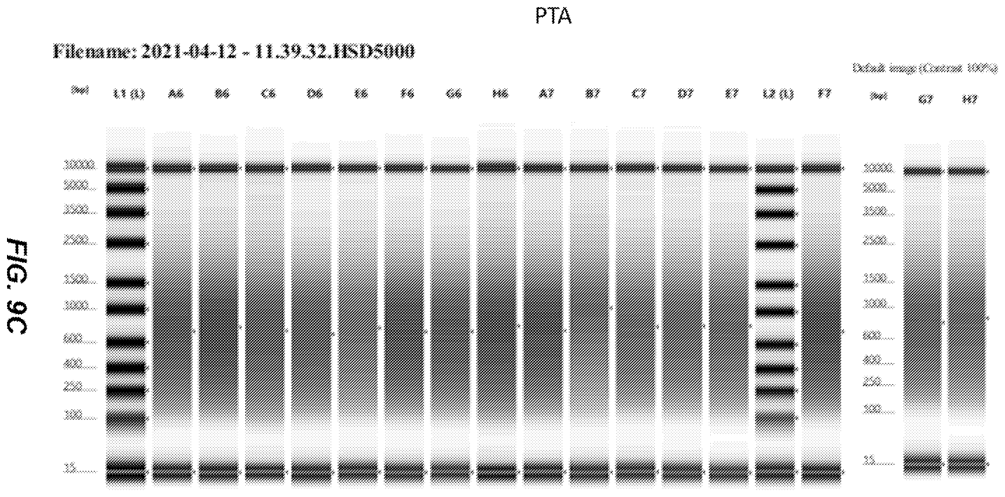

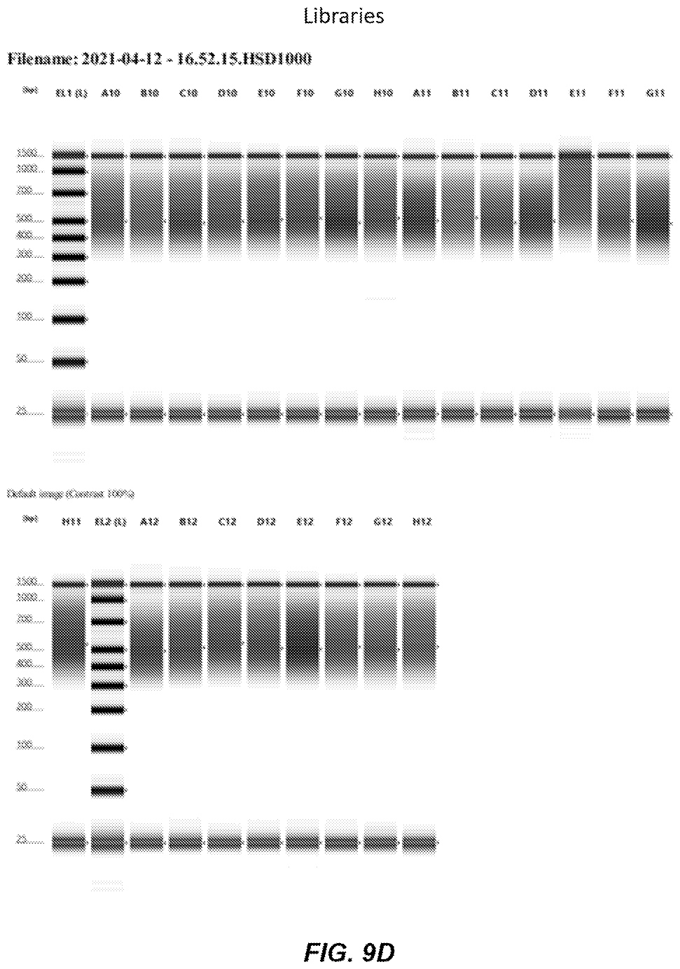

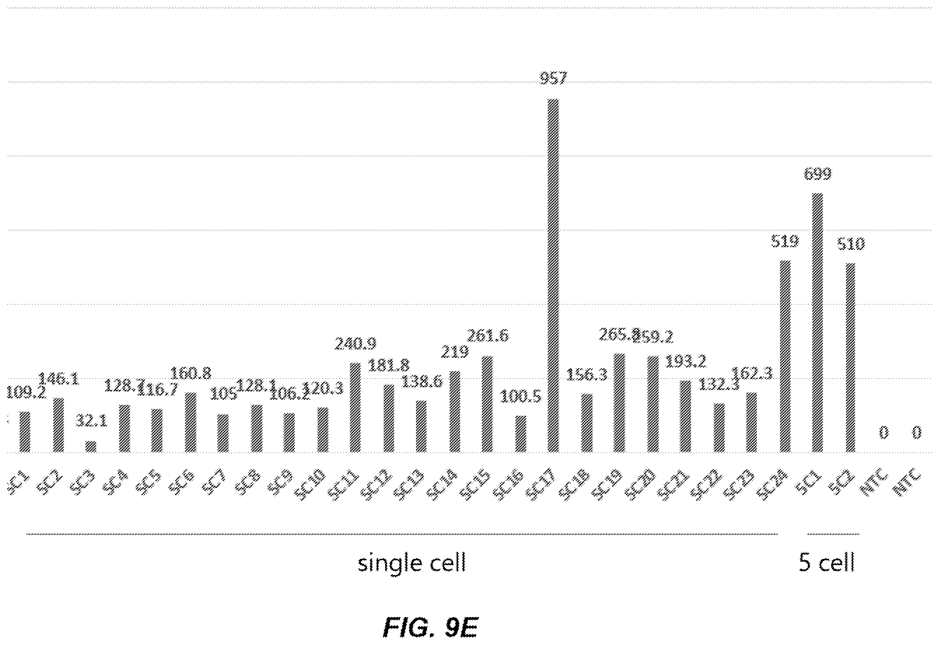

Following the general methods of Examples 1-3, B. subtilis was analyzed using the PTA method. Free-dried cultures were obtained from the ATCC and cultured for 48 hours before staining and FACS sorting. Results are shown in FIGS. 9A-9B. After isolation of single (or groups of 5 cells, similar to Example 2), these cells were subjected to PTA amplification conditions. Size distributions from PTA and the library are shown in FIG. 9C and FIG. 9D, respectively. Yields of amplified DNA from PTA are shown in FIG. 9E.

### Example 5: Detection of Mixed Populations of Bacteria

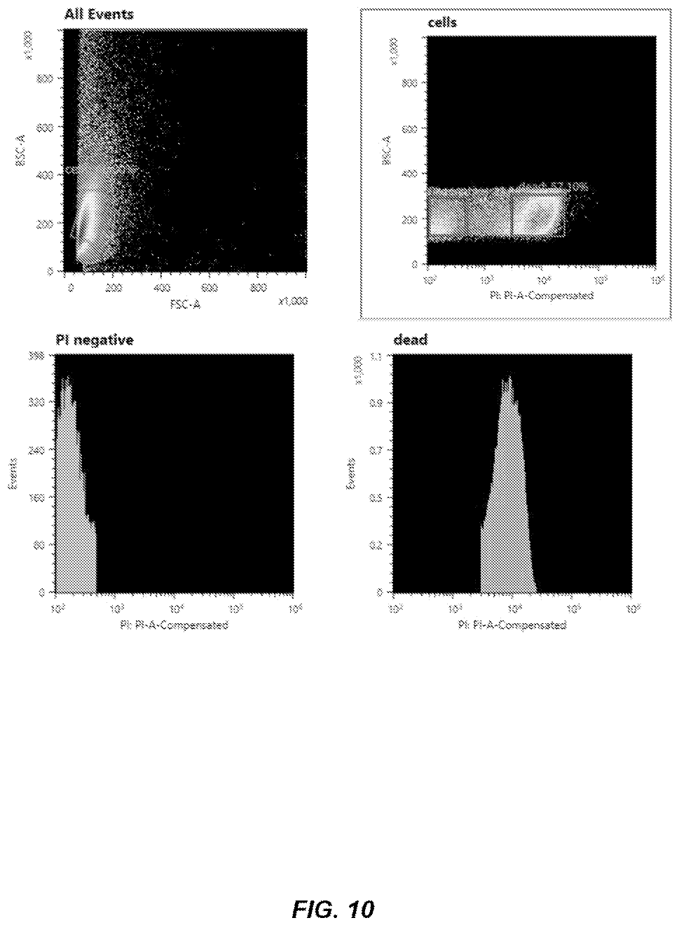

The general methods of Examples 1-4 were followed with modification: a mixture of 1.5:1 B. subtilis: E. coli was prepared just prior to FACS sorting. The sorting data is shown in FIG. 10, and can be analyzed using the PTA method.

### Example 6: PTA for Trace Nucleic Acid Detection

Nucleic acid contamination may interfere with highly sensitive manufacturing processes which are commonly carried out in ISO-certified clean rooms. Additionally, analysis of contaminating nucleic acids, if found, may provide guidance on identifying sources of contamination. Samples (96) are obtained from a source (such as the surface of various instruments in a clean room) to obtain nucleic acids for parallel analysis. The samples are individually contacted with a buffer, and a portion of the sample is combined with an amplification mix (randomized primers of 8-15 nt in length, dNTPs, Phi29 polymerase, buffer) that further contain 10% alpha-thio-ddNTPs (relative to dNTPs) in a 96 well plate. The samples are then placed at 30° C. for 8 hours after which the amplification is terminated by heating to 65° C. for 3 minutes.

After the amplification step, the DNA from the PTA reaction is purified using AMPure XP magnetic beads at a 2:1 ratio of beads to sample. A fluorescence signal is obtained for each sample which correlates with the concentration of nucleic acids, which is then compared to a pre-established signal threshold. Samples exceeding the threshold test positive for nucleic acids. Pre-established thresholds may be obtained by generating a signal to noise ratio for various concentrations of nucleic acids in prior experiments. Sample testing positive for nucleic acids may be further quantified, or subjected to library generation and next generation sequencing for additional analysis. In some instance, the entire nucleic acid detection method is automated.

### Example 7: DNA Detection for Reagent Manufacturing

Biotechnology reagents, especially those used for nucleic acid-based diagnostic tests, require high purity from nucleic acid contamination. A host organism, such as E. coli, is transformed with a plasmid which allows for recombinant expression of an enzyme, such as a polymerase. The enzyme is purified using standard workflows (e.g., ion chromatography, affinity chromatography, size exclusion, etc.), and a sample of the purified product is tested for nucleic acid contamination using the general methods of Examples 1 and 6. If the level of nucleic acids in the sample is above a predetermined threshold, the purified product is discarded or re-purified. In some instances, organism-specific primers are used for PTA amplification instead of random primers, such as primers where are configured to bind to portions of the E. coli genome.

### Example 8: Pathogen Detection of Campylobacter in Poultry Samples

Campylobacter is a food-borne pathogen contamination commonly found in raw or undercooked poultry, which may produce serious illness in humans. Samples are obtained from boot and carcass swabs in a poultry manufacturing plant. These samples are processed using the general methods of Examples 1 and 6, with modification; target-organism specific primers are used to amplify portions of the Campylobacter genome. These primers comprise a reporter moiety and a quencher moiety tether by a cleavable linker, such that successful amplification of the target organism genome results in a fluorescent signal (e.g., quencher is cleaved by an exonuclease, such as exonuclease III). The signals are measured, compared with pre-determined threshold levels, and each sample is determined to be positive or negative for Campylobacter contamination.

### Example 9: Forensic Analysis

Nucleic acid analysis is a common method of obtaining information pertaining to ongoing criminal investigations. However, the time required to obtain and analyze high-quality samples may slow such investigations. Following the general procedures of Examples 1 and 6, 384 samples are obtained from areas in a suspected crime scene. These samples are subjected to PTA with modification using an in-field kit; a pH-sensitive dye is added along with the other PTA reagents. The extent of amplification (and therefore nucleic acid concentration) is visible as a change in color, which allows an investigator to rapidly prioritize samples having higher (or at least some) DNA concentrations for sequencing. In some instance, primers configured to bind to human genomic DNA are used instead of random primers.

### Example 10: Real-Time PCR Monitoring of Clinical Isolate

A clinical bacterial isolate sample is obtained from a patient and analyzed using the general procedures of Examples 1 and 6, with modification. Three different fluorescent primer probes are used to monitor the amplification reaction in real time. Each primer probe is configured to bind to a specific regions of three different alleles found in a multi-drug resistant bacterial strain, and each probe produces a readable signal at a different wavelength. Signals are obtained for each primer probe at various time points during the amplification reaction. Analysis of the quasi-exponential portion of the amplification reaction as a function of time is used to simultaneously quantify the amount of each allele identified. Using this information, specific antibiotics are administered that will be most effective in treating the bacteria.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of detecting the presence or absence of trace nucleic acids comprising:
a. providing a sample from a source, wherein the source comprises no more than 1 nanogram of nucleic acids;
b. contacting the sample with at least one amplification primer, at least one strand displacement polymerase, and a mixture of nucleotides to generate replication products; and
c. measuring a signal obtained from the replication products, wherein the signal to a noise ratio (SNR) greater than 1.01 indicates the sample comprises trace nucleic acids.

2. The method of claim 1, wherein the sample comprises no more than 0.1 nanograms of nucleic acids.

3. The method of claim 1, wherein the sample comprises no more than 1 picograms of nucleic acids.

4. The method of claim 1, wherein the sample comprises no more than 10 femtograms of nucleic acids.

5. The method of claim 1, wherein the nucleic acids comprise no more than 50 million nucleosides.

6. The method of claim 5, wherein the nucleic acids comprise no more than 100,000 nucleosides.

7. The method of claim 5, wherein the nucleic acids comprise no more than 10,000 nucleosides.

8. The method of any one of claims 1-7, wherein the nucleic acids have an average length of 200-2000 bases.

9. The method of any one of claims 1-7, wherein the nucleic acids have an average length of at least 1000 bases.

10. The method of any one of claims 1-9, wherein the method further comprises establishing a noise amount from a no template control experiment.

11. The method of any one of claims 1-10, wherein contacting occurs for no more than 10 hours.

12. The method of any one of claims 1-10, wherein contacting occurs for no more than 4 hours.

13. The method of any one of claims 1-10, wherein contacting occurs for no more than 2 hours.

14. The method of any one of claims 1-13, wherein the ratio of the signal to the noise is greater than 1000.

15. The method of claim 14, wherein a SNR greater than 1.05 indicates the sample comprises trace nucleic acids.

16. The method of claim 14, wherein a SNR greater than 1.1 indicates the sample comprises trace nucleic acids.

17. The method of any one of claims 1-15, wherein the signal is fluorescence, phosphorescence, chemiluminescence, or colorimetric.

18. The method of any one of claims 1-17, wherein the nucleic acids comprise nucleic acids derived from bacteria, yeast, fungi, molds, insect, or human sources.

19. The method of claim 18, wherein the sample is obtained from one or more of an enzyme-containing reagent, a pharmaceutical composition, a boot or carcass swab, blood, hair, skin, saliva, and a human clinical isolate.

20. The method of claim 18, wherein the sample further comprises proteins.

21. The method of claim 19, wherein the proteins are recombinantly expressed proteins.

22. The method of any one of claims 1-21, wherein the sample comprises at least one nucleic acid, and the at least one nucleic acid is amplified in step b).

23. The method of claim 22, wherein the amplification is performed under substantially isothermic conditions.

24. The method of claim 23, wherein the amplification is performed under conditions wherein the temperature varies by no more than 10 degrees C.

25. The method of claim 23, wherein the amplification is performed under conditions wherein the temperature varies by no more than 5 degrees C.

26. The method of any one of claims 1-25, wherein the nucleic acid polymerase is a DNA polymerase.

27. The method of claim 26, wherein the DNA polymerase is a strand displacing DNA polymerase.

28. The method of claim 26, wherein the nucleic acid polymerase is bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, Vent_{R} DNA polymerase, Vent_{R} (exo-) DNA polymerase, Deep Vent DNA polymerase, Deep Vent (exo-) DNA polymerase, IsoPol DNA polymerase, DNA polymerase I, Therminator DNA polymerase, T5 DNA polymerase, Sequenase, T7 DNA polymerase, T7-Sequenase, or T4 DNA polymerase.

29. The method of any one of claims 1-28, wherein the nucleic acid polymerase does not comprise 3′->5′ exonuclease activity.

30. The method of claim 29, wherein the polymerase is Bst DNA polymerase, exo(−) Bst polymerase, exo(−) Bca DNA polymerase, Bsu DNA polymerase, Vent_{R} (exo-) DNA polymerase, Deep Vent (exo-) DNA polymerase, Klenow Fragment (exo-) DNA polymerase, or Therminator DNA polymerase.

31. The method of any one of claims 1-30, wherein the mixture of nucleotides comprises at least one terminator nucleotide which terminates nucleic acid replication by the strand displacement polymerase.

32. The method of claim 31, wherein the nucleic acid polymerase comprises 3′->5′ exonuclease activity and the at least one terminator nucleotide inhibits the 3′->5′ exonuclease activity.

33. The method of claim 31, wherein the least one terminator nucleotide comprises modifications of the r group of the 3′ carbon of the deoxyribose.

34. The method of claim 31, wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof.

35. The method of claim 31, wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

36. The method of claim 31, wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

37. The method of claim 31, wherein the nucleotides with modification to the alpha group are alpha-thio dideoxynucleotides.

38. The method of claim 31, wherein the amplification primers are 4 to 70 nucleotides in length.

39. The method of claim 31, wherein the at least one amplification primer is 4 to 20 nucleotides in length.

40. The method of claim 38 or 39, wherein the at least one amplification primer comprises a randomized region.

41. The method of claim 40, wherein the randomized region is 4 to 20 nucleotides in length.

42. The method of claim 40 or 41, wherein the randomized region is 8 to 15 nucleotides in length.

43. The method of any one of claims 1-42, wherein the amplification products are between about 50 and about 2000 nucleotides in length.

44. The method of any one of claims 1-43, wherein the amplification products are between about 200 and about 1000 nucleotides in length.

45. The method of any one of claims 1-43, wherein the amplification proceeds for 5-15 cycles.

46. The method of any one of claims 1-43, wherein the amplification proceeds for no more than 20 cycles.

47. The method of any one of claims 1-46, wherein the method further comprises qPCR.

48. The method of any one of claims 1-46, wherein at least one amplification primer comprises a cleavable fluorophore and quencher.

49. The method of any one of claims 1-46, wherein the method comprises at least four amplification primers.

50. The method of any one of claims 1-46, wherein the method further comprises contacting the sample with a single-stranded DNA binding protein.

51. The method of any one of claims 1-46, wherein the method further comprises contacting the sample with a helicase.

52. The method of any one of claims 1-46, wherein the method further comprises contacting the sample with a nicking enzyme.

53. The method of any one of claims 1-46, wherein the method further comprises contacting the sample with a reverse transcriptase.

54. The method of any one of claims 1-53, wherein the method further comprises quantifying the concentration of nucleic acids in the sample.

55. The method of any one of claims 1-53, wherein the method further comprises discarding or repurifying a sample which is found to contain trace nucleic acids.
